(19) United States
(12) Patent Application Publication
(10) Pub. No.: US 2010/0173355 A1
(43) Pub. Date: Jul. 8, 2010

(54) Means for sequestration and conversion of COx and NOx, CONOx

(75) Inventors: Richard Alan Haase, Missouri City, TX (US); Fadhil Salih, Spring, TX (US); Candice Marie Haase, Missouri City, TX (US)

Correspondence Address: RICHARD A. HAASE (INVENTOR), 4402 RINGROSE DRIVE, MISSOURI CITY, TX, 77459, US

(73) Assignee: ClearValue Technologies, Inc., Missouri City, TX (US)

(21) Appl. No.: 12/660,945

(22) Filed: Mar. 8, 2010

(51) Int. Cl. C12P 1/00

(52) U.S. Cl. 435/41

(57) ABSTRACT

The instant invention presents means for sequestering CO_{X} and NO_{X}; further comprising algae means to convert CO_{X} into oxygen (O₂), as well as biological means to convert sulfides into elemental sulfur. The instant invention comprises algae, heterotrophs, facultative bacteria and Thiobacillus. The instant invention comprises means of light (photon) transfer. Fiber optics is a means of photon transfer to provide photons to a biological reactor. The instant invention comprises the photon depth adsorption capability of algae in biological reactor means. The instant invention comprises means of energy management so that the instant invention may be used in most any environment, wherein a photon (light) source is available and can comprise a means of photon source generation when a light source is not available. The instant invention is an economical means of hydrocarbon production.

### RELATED APPLICATION DATA

This application claims priority on PCT/US08/010,495 filed Sep. 6, 2008; U.S. patent application Ser. No. 12/231,992 filed Sep. 8, 2008; U.S. Provisional Application 60/967,742 filed Sep. 6, 2007; U.S. Provisional Application 61/011,403 filed Jan. 17, 2008; and U.S. Provisional Application 61/130,706 filed Jun. 2, 2008.

### BACKGROUND OF THE INVENTION

1. Field of the Invention

The instant invention relates to improved means (herein means is defined as at least one of a method, processes and apparatus) for the sequestering of oxides of carbon and oxides of nitrogen. The instant invention improved means for the scrubbing of oxides of carbon and oxides of nitrogen is herein defined as the Hydrocarbon combustion Aqueous Assimilation System for the Environment (HAASE). HAASE chemically assimilates at least one of: oxide(s) of carbon (CO and CO₂, herein after referred to as CO_{X}), and oxide(s) of nitrogen (N_{Y}O_{X}, which can be N₂O, NO, NO₂ or NO₃ and are herein after referred to as NO_{X}) from a hydrocarbon combustion gas. Within the instant invention, Gas Flow is defined as a source and/or flow of gas comprising CO_{X} and/or NO_{X}.

The instant invention (HAASE) relates to a means for minimizing CO_{X} and/or NO_{X} emissions. The instant invention (HAASE) relates to reducing and/or minimizing CO_{X} and/or NO_{X} emissions emanating from the burning of fossil fuels or extracting natural gas or of converting a hydrocarbon into hydrogen (H₂).

The instant invention further comprises algae means of converting CO_{X} into oxygen (O₂). The instant invention comprises sulfur consuming bacteria means, most preferably of the genus Thiobacillus, to convert sulfides into elemental sulfur. The instant invention comprises heterotrophic bacteria means to purify water of hydrocarbons. The instant invention comprises algae, heterotrophs, facultative bacteria and Thiobacillus as means of converting NO_{X} into N₂.

The instant invention comprises means of light (photon) transfer. Fiber optics is a means of photon transfer for the instant invention to provide photons to a biological reactor. The instant invention comprises translucent materials, most preferably those made of silicon or of carbonate, as biological reactor means and photon transport from fiber optics to the biological reactor. The instant invention comprises the photon depth adsorption capability of algae in biological reactor means. The instant invention comprises means of energy management so that the instant invention may be used in most any environment, wherein a photon (light) source is available and can comprise a means of photon source when a light source is not available.

The instant invention comprises a means of O₂ and of hydrogen (H₂) production. The instant invention comprises both O₂ and H₂ production capabilities of algae.

Currently, humanity has significant interest in reducing CO_{X} and NO_{X} gas emissions into the atmosphere. The amount of CO_{X} emitted into the atmosphere is cited as a factor contributing to global climate change and acidification of Earth's Oceans from dissolved carbonic acid. CO_{X} is emitted whenever fossil fuels are burned. NO_{X} is emitted whenever fossil fuels are burned with air or with nitrogen (N₂) in combustion, such as in automobile engines and fossil fuel burning furnaces or boilers. Reducing CO_{X} and NO_{X} emissions is of increased importance to humanity and is a point of emphasis for government regulatory agencies. Further, humanity is in search of new energy sources; as, sources of liquid hydrocarbons, e.g. oil, are being depleted.

2. Background of the Invention

Mankind has, over the centuries, developed many forms of energy, along with many forms of transportation. In the modern economy, energy is needed to literally “fuel” the economy. Energy heats homes, factories and offices; provides electrical power; powers manufacturing facilities, and provides for the transportation of goods and people.

During the 19'th and 20'th centuries, mankind developed fossil, hydrocarbon, fuels into reliable and inexpensive energy sources; this is while fossil fuel combustion releases polluting compounds into the air, some of which pollute waters. The combustion products of fossil fuels have become a major source of air and water (H₂O) pollution.

Fossil fuels (hydrocarbons) are used as a fuel along with air as an oxidant to generate combustion energy. Hydrocarbons, C_{X}H_{Y}, are most often either: petroleum distillates such as gasoline, diesel, fuel oil, jet fuel and kerosene; or, fermentation distillates such as methanol and ethanol; or, natural products such as methane, ethane, propane, butane, coal and wood. The products of hydrocarbon combustion were thought to work in concert with nature's O₂-carbon cycle, wherein CO₂ is recycled by plant life photosynthesis back into O₂. However, excess hydrocarbon combustion interferes with nature; excess CO_{R} in the atmosphere upsets the environment causing global climate change. The combustion of a hydrocarbon can be approximated by:

CₙH₂ₙ₊₂+( 3/2n+½)O₂→nCO₂+(n+1)H₂O+Energy

More specifically, for gasoline (2,2,4 trimethyl pentane or Octane):

gasoline (octane)+12½O₂→8CO₂+9H₂O+1,300 kcal

And, for natural gas (methane):

CH₄+ 3/2O₂→CO₂+2H₂O+213 kcal

So, CO_{X} is produced by the combustion of fossil fuels, while global climate change is a result of a buildup of CO_{X} in the Earth's atmosphere. And, while photosynthesis will naturally turn CO₂ back into O₂, man-made production of CO₂ in combination with significant deforestation have left earth's plant life incapable of converting enough of manmade CO₂ back into O₂. This is while CO, an incomplete combustion by-product, is toxic to all human, animal and plant life.

In addition, hydrocarbon combustion with air creates NO_{X}; NO_{X} retards photosynthesis while being toxic to all human, animal and plant life. Once formed, NO_{X} further reacts with O₂ in the air to form ozone (O₃). O₃ is toxic to all human, animal and plant life. O₃ does protect the earth in the upper atmosphere from harmful solar UV radiation; however, at the Earth's surface O₃ is toxic. Therefore, the production of NO_{X} further interferes with the capability of earth's plant life to convert enough of manmade CO₂ back into O₂.

Lastly, CO_{X} and NO_{X} react with H₂O in the air and on the Earth's surface to form acids, e.g. H₂CO₃, HNO₂ and HNO₃, which in the air, then, literally rain acids upon the earth.

Hydrocarbon fuels have been modified with additives to minimize the formation of either CO_{X} or NO_{X}. However, with all of the engine modifications and fuel modifications, the Earth has become unable to keep up.

In the instant invention, Gas flow is defined as any flow of a gas which comprises CO_{X}, and may further comprise at least one of: NO_{X}, S_{X}, any metal oxide, and any combination therein. Gas flow may have any origination. Gas flow is preferably from at least one of a combustion source and a source of hydrocarbon fuel(s).

It is known in general chemistry to react CO_{X}, with an aqueous solution comprising at least one of: sodium hydroxide (NaOH), potassium hydroxide (KOH), calcium hydroxide (Ca(OH)₂), magnesium hydroxide (Mg(OH)₂), and any combination therein to form a solid precipitate of carbonate (CO₃²⁻) or of bi-carbonate (HCO₃⁻) with the corresponding metal cation. However, these means suffer from either the use of a hazardous chemical, e.g. NaOH or KOH, or a chemical which is difficult to keep soluble, e.g. Ca(OH)₂ or Mg(OH)₂. Processes for the adsorption of CO₂ with a group IA and IIA metal hydroxide are disclosed and presented in U.S. Pat. No. 4,407,723, while used as a reference in this instant invention.

It is known in general chemistry to react NO_{X} in water to form nitrite (NO₂⁻) or nitrate (NO₃⁻) and then react the NO₂⁻ or NO₃₋ with ammonia (NH₃) or aqueous ammonium (NH₄OH) to form ammonium nitrate (NH₄NO₃); however, NH₄NO₃ is also a hazardous chemical, especially when exposed to a hydrocarbon or fossil fuel.

Currently, systems for controlling and eliminating CO₂ from a breathable air supply are utilized in submarines, space vehicles and space suits. These systems utilize a CO₂ sorbent bead composed of a plurality of amine sorbent beads disposed within a container. A stream of air containing CO₂ is flowed through the container and the amine sorbent beads. The CO₂ contacting the amine sorbent beads react therewith to become trapped within the container. The remainder of the breathable air recirculates into the controlled environment. Once the container has become saturated with CO₂, such that further absorption of CO₂ is inefficient, the breathable air stream is switched to a second container. The saturated container is then exposed to heat or reduced pressure to evolve or release the trapped CO₂ for disposal or use in other systems. Such systems have proven effective and efficient for controlling the CO₂ content within an enclosed environment; however, this technology and related technologies still must release CO₂. Processes for the adsorption of CO₂ are disclosed and presented in U.S. Pat. Nos. 2,545,194; 3,491,031; 3,594,983; 3,738,084; 3,939,068; 4,005,708; 4,233,175; 4,407,723; 4,426,364; 4,539,189; 4,668,255; 4,674,309; 4,810,266; 4,822,383; 4,999,175; 5,281,254; 5,376,614; 5,462,908; 5,492,683; 5,518,626; 5,682,709; 5,770,785; 5,876,488; 6,274,108; 6,355,094; 6,364,928; 6,547,854; 6,755,892; 6,890,497; 7,247,285 and U.S. Publication 2002/0083833, while all are used as a reference in this instant invention.

Previous work in the scrubbing of hydrocarbon combustion gases focused on the removal of oxides of sulfur (SO_{X}) by reaction of SO_{X} with an alkaline earth metal in order to form a calcium sulfate. Processes for the adsorption of SO_{X} are disclosed and presented in U.S. Pat. Nos. 4,233,175 and 7,247,285, while used as a reference in this instant invention.

Current catalyst work to convert NO_{X} to N₂ comprises reacting the NO_{X} with platinum and rhodium catalyst. This type of catalysis is commonly used in the three-way catalytic converters in transportation applications. Also, current work to transport and/or store CO_{X} comprises compression of the CO_{X} gas, as well as the underground compression and eventual liquefaction of the CO_{X} gas. This underground storage and/or liquefaction presents many costs and risks; as, there is a significant energy requirement to compress and transfer the CO_{X} gas and there is a risk that underground storage of the CO_{X} gas may leak to the Earth's Surface. Finally, current work in concert with this application, U.S. patent application Ser. No. 12/231,992 is incorporated herein by reference.

Hydrogen Combustion—The instant invention produces O₂ and H₂. The instant invention embodies combustion as an energy source for the instant invention, wherein the fuel comprises H₂ and the oxidizer comprises O₂. The instant invention minimizes the use of N₂ in combustion so as to limit NO_{X} formation. Previous work presented in these means can be found in PCT/US03/11250; PCT/US 03/041719; and PCT/US06/048057, all of which are incorporated herein by reference.
Water Dispersion Chemistry—The instant invention relates to means of controlling COx and NOx scale and deposition in water applications. U.S. Pat. No. 4,209,398 issued to Ii, et al., on Jun. 24, 1980, while used as a reference in this instant invention, presents a process for treating water to inhibit formation of scale and deposits on surfaces in contact with the water and to minimize corrosion of the surfaces. The process comprises mixing in the water an effective amount of water soluble polymer containing a structural unit that is derived from a monomer having an ethylenically unsaturated bond and having one or more carboxyl radicals, at least a part of said carboxyl radicals being modified, and one or more corrosion inhibitor compounds selected from the group consisting of inorganic phosphoric acids and water soluble salts thereof, phosphonic acids and water soluble salts thereof, organic phosphoric acids and water soluble salts thereof, organic phosphoric acid esters and water-soluble salts thereof and polyvalent metal salts, capable of being dissociated to polyvalent metal ions in water. The Ii patent does not discuss or present systems of COx and/or NOx sequestration. U.S. Pat. No. 4,442,009 issued to O'Leary, et al., on Apr. 10, 1984, while used as a reference in this instant invention, presents a method for controlling scale formed from water soluble calcium, magnesium and iron impurities contained in boiler water. The method comprises adding to the water a chelant and water soluble salts thereof, a water soluble phosphate salt and a water soluble poly-methacrylate acid or water soluble salt thereof. The O'Leary patent does not discuss or present systems of COx and/or NOx sequestration.

U.S. Pat. No. 4,631,131 issued to Cuisia, et al., on Dec. 23, 1986, while used as a reference in this instant invention, presents a method for inhibiting formation of scale in an aqueous steam generating boiler system. Said method comprises a chemical treatment consisting essentially of adding to the water in the boiler system scale-inhibiting amounts of a composition comprising a copolymer of maleic acid and alkyl sulfonic acid or a water soluble salt thereof, hydroxylethylidene, 1-diphosphic acid or a water soluble salt thereof and a water soluble sodium phosphate hardness precipitating agent. The Cuisia patent does not discuss or present systems of COx and/or NOx sequestration. U.S. Pat. No. 4,640,793 issued to Persinski, et al., on Feb. 3, 1987, while used as a reference in this instant invention, presents an admixture, and its use in inhibiting scale and corrosion in aqueous systems, comprising: (a) a water soluble polymer having a weight average molecular weight of less than 25,000 comprising an unsaturated carboxylic acid and an unsaturated sulfonic acid, or their salts, having a ratio of 1:20 to 20:1, and (b) at least one compound selected from the group consisting of water soluble polycarboxylates, phosphonates, phosphates, polyphosphates, metal salts and sulfonates. The Persinski patent presents chemical combinations which prevent scale and corrosion; however, the Persinski patent does not discuss or present systems of COx and/or NOx sequestration.

Sulfur Consuming Bacteria—In recent years, there have been identified many species (sp.) of bacteria which metabolize or consume sulfur in their biomass. Most of these bacteria are obligate aerobes capable of taking oxygen, SO₂, SO₃, NO₃, and NO₃ as an electron donor source for the conversion of S_{X} to Sulfur (S). Most of these bacteria have difficulty or react slowly to convert SO₄ to S. Many of these bacteria are capable of operating in an aerobic environment. An aerobic environment is not preferred as in an aerobic environment a portion of the sulfides are converted to sulfate, which converts to sulfuric acid. Therefore, facultative or anoxic bacteria in an anoxic environment are preferred in the conversion of sulfides to S so as to minimize the formation of sulfate.

Bacteria known for their conversion of sulfides to elemental sulfur in their biomass include but are not limited to species of the genus Thiobacillus and the species therein of Thiobacillus denitrificans most known and as presented in U.S. Pat. No. 6,126,193 and U.S. Pat. No. 5,705,072, both of which are referenced to in the instant invention; gram-negative bacteria from the beta or gamma subgroup of Proteobacteria, obligate autotrophs, Thioalkalovibrio strain Al-2, Thioalkalobacter, alkaliphilic heterotrophic bacteria, and Pseudomonas strain ChG 3, all of which as described in U.S. Pat. No. 6,156,205, while used as a reference in this instant invention. Further strains are described in U.S. Pat. No. 7,101,410, while used as a reference in this instant invention, lists: Rhodococcus eythropolis, Rhodococcus rhodochrous, other Rhodococcus sp., Nocardia erythropolis, Nocardia corrolina, other Nocardia sp., Pseudomonas putida, Pseudomonas oleovorans, other Pseudomonas sp., Arthrobacter globiformis, Arthobacter Nocardia paraffinae, Arthrobacter paraffineus, Arthrobacter citreus, Arthrobacter luteus, other Arthrobacter sp., Mycobacterium vaccae JOB and other species of Mycobacterium Acinetobacter and other species of Acinetobacter, Corynebacterium and other Corynebacterium sp., Thiobacillus ferrooxidans, Thiobacillus intermedia, other species of Thiobacillus shewanella, Micrococcus cinneabareus, other micrococcus sp., Bacillus sulfasportare and other bacillus sp. Fungi, White wood rot fungi, Phanerochaete chrysosporium, Phanerochaete sordida, Trametes trogii, Tyromyces palustris, other white wood rot fungal sp., Streptomyces fradiae, Streptomyces globisporus, and other Streptomyces sp., Saccharomyces cerrevisiae, Candida sp., Cryptococcus albidus, yeasts and algae.

Denitrifying Bacteria—It has heretofore been well known that existence of nitrogen compounds is one cause of river and lake eutrophication. In the biological treatment of water, ammonia nitrogen contained in for-treatment water is converted into NO₃⁻. Then the NO₃⁻ can be reduced to N₂ gas by denitrifying bacteria. This reduction is brought about by certain bacteria which are able, in the absence of O₂, to utilize NO₃⁻ and NO₂⁻ in place of O₂ to oxidize available and microbially utilizable organic compounds. In the chemical reaction characterized by this microbial process, NO₃⁻ and NO₂⁻ serve as terminal electron donors and the assimilable or microbially utilizable carbon compounds serve as electron acceptors. Since the purpose of microbial denitrification is to eliminate all oxidized nitrogen compounds, it is essential that there be available an excess of the carbon/energy source to insure that denitrification goes to its theoretical completion and that there be sufficient additional carbon available for bacterial growth. The amount of carbon required can be readily calculated stoichiometrically and where methanol is the carbon source, 3.0 mg/l of methanol will adequately reduce 1 mg/l of NO₃⁻ and provide sufficient carbon for bacterial growth.

Carbon source supplementation is essential to compensate for carbon and BOD deficiencies in both the digested nitrocellulose waste and the domestic sewage. Denitrification can be carried out in a conventional tank of suitable size using activated sludge or wastewater as a source of suitable denitrifying bacteria or relying on the bacteria normally present in raw sewage and holding the mixed liquor under essentially anaerobic conditions. The time required for denitrification will depend on the concentration of NO₃⁻ and NO₂⁻, the temperature of the liquor within the tank, the dissolved oxygen content, the population of denitrifying bacteria and the concentration of available microbially utilizable carbon material. None of the foregoing conditions is critical except that the dissolved O₂ concentration must be below that normally required for aerobic microbial growth and the temperature of the liquor should not drop below that at which the bacteria can efficiently denitrify the NO₃⁻ and NO₂⁻. Many common facultative bacteria are able to effect denitrification, including members of the genera Pseudomonas, Bacillus, and Achromobacter, as well as the facultative specie of Thiobacillus, such as Thiobacillus denitrificans. Suitable denitrifying bacteria will be present in most activated sludge mass material or raw sewage material. After denitrification is completed, solids in the liquor are allowed to settle either in the same vessel or in a separate sedimentation vessel. Following sedimentation, the clear effluent is removed and the solids remaining are recycled for further denitrification. While these microbial processes are well known, there is no currently means of employing these means in the conversion of NO_{X} gas.

It is well known in biology that algae will convert CO₂ into O₂ using light (photons) as an energy source in CO₂ Conversion. What has been recently discovered is the efficiency with which CO₂ Conversion is performed. Algae are near 20 to 25 times more efficient, on a mass basis, as plants in converting CO₂ into O₂. In addition, it has recently been discovered that many species of algae are capable of H₂ production in the absence of O₂, wherein at least one of S and N₂ are removed from the algal environment.

Algae Biological Reactor (ABR)—Recent attempts in means for an algal biological reactor (ABR) to perform CO₂ Conversion (herein CO₂ Conversion is defined as the algal conversion of CO₂ to O₂) incorporate either a film growth of algae or the growth of algae in polycarbonate tubes. Previous work in ABR development is presented and referenced herein in U.S. Pat. Nos. 6,056,919; 6,083,740; 6,199,317; 6,237,284; 6,287,852; 6,395,521; 6,410,258; 6,648,949; 7,191,736; and in Masojidek, J., et al., A Closed Solar Photobioreactor for Cultivation of Microalgae Under Supra-high Irradiance: Basic Design and Performance, Journal of Applied Phycology 15: 239-248, 2003; Akira Satoh, et al. Effects of Chloramphenicol on Photosynthesis, Protein Profiles and Transketolase Activity under Extremely High CO₂ Concentration in an Extremely-high-CO₂-tolerant Green Microalga, Chlorococcum littorale, Marine Biotechnology Institute, 3-75-1 Heita, Kamaishi, Iwate, 026-0001 Japan; Jaffe S., Mutant Algae Is Hydrogen Factory, http://www.wired.com/science/discoveries/news/2006/02/70273; Kremer, G., Practical Photosynthetic Carbon Dioxide Mitigation, Ohio Coal Research Center, www.ent.ohiou.edu˜ohiocoal; Sheehan, J. et al., A Look Back at the U.S. Department of Energy's Aquatic Species Program—Biodiesel from Algae, National Renewable Energy Laboratory, 1998; Yusuf, Chisti, Biodiesel from Microalgae, Biotechnology Advances 25, 294-306, 2007; Jeong, Mijeong J., et al., Carbon Dioxide Mitigatin by Micralgal Photosynthesis, Korean Chemical Society, Vol. 24 No. 12, 1763, 2003; Sobczuk, T. Mazucca, et al., Carbon Dioxide Uptake Efficiently by Outdoor Microalgal Cultures in Tubular Airlift Photobioreactors, Department of Chemical Engineering University of Almeria E-04071 Almeria, Spain, John Wiley and Sons, 2003; and Gavis, Jerome and Ferguson, John F., Kinetics of Carbon Dioxide Uptake by Phytoplankton at High pH, all of which are incorporated herein by reference. These means are deficient in space utilization, materials of construction and energy management. It is especially worth noting that the '949 patent specifically minimizes and/or limits carbonate precipitation; such a limitation would lead to rather large vapor scrubbing operations, along with the management of significant volumes of water. Film growth of algae, while effective, requires a significant amount of space to place the algal film and algal film support media. Polycarbonate as a material is inherently deficient in its ability to withstand photon polymer degradation. Finally, energy management means is needed so that CO₂ Conversion may be performed in colder climates, as well as temperate climates.
Optical Fibers—The instant invention relates to means of photon (light) transfer. The instant invention relates to means of fiber optics, as well as tubular optics. The instant invention teaches the use of fiber optic cable as a means to transfer light (photons) to an ABR. Previous work presented in these means can be found in U.S. Pat. Nos. 4,877,306; 5,212,757; 6,316,516; and 7,088,897, all of which are incorporated herein by reference.
Diffusion—The instant invention relates to means of gas transfer (diffusion) into a liquid. The instant invention teaches fine bubble diffusion of CO₂ and NO_{2 or 3} into water. Previous work in this art can be found in U.S. Pat. Nos. 4,960,546; 5,015,421; 5,330,688; 5,676,890; 6,464,211; 7,311,299, all of which are incorporated herein by reference.
Liquid/Solids Separation—The instant invention relates to means of separating algae from water and in the dewatering of algae. Previous work in this art can be found in U.S. Pat. Nos. 6,120,690; 5,846,435; and 5,906,750 and U.S. Pat. Publication 2003/029499, all of which are incorporated herein by reference.

As humanity battles global climate change, a long felt and unresolved need exists for new energy sources, a means of managing hydrocarbon combustion emissions, especially those from a power plant or a hydrocarbon source such as a natural gas well or a coal gasification plant, specifically CO_{X} and NO_{X} emissions. While algae appear to the in the solution mix for these significant long felt and unresolved human needs, humanity still searches for a practical means to use algae. Also, there exist a significant long felt and unresolved need for a means to manage an ABR regardless of ambient temperature and with minimal equipment and a reduction in space utilization.

In summary, CO_{X}, NO_{X} and O₃ are direct, indirect and resultant products, respectively, of the combustion of hydrocarbons. These products adversely affect: all life, our environment and health of our Earth. The instant invention has proven an environmentally acceptable method, process or apparatus to significantly reduce the concentration of CO_{X} and/or NO_{X}, especially from hydrocarbon combustion while creating a salt which works in concert with and occurs regularly in nature. This is while there is a significant and here-to-fore unmet and long felt need of humanity to sequester and preferably convert CO_{X} and/or NO_{X} gases.

The instant invention has surprisingly been found as a means of ABR which provide humanity an efficient and effective means of CO₂ Conversion, wherein space utilization is near optimal or significantly improved, materials of construction are improved and energy management is obtained, regardless of ambient temperature. The instant invention is surprisingly found to be an answer to the aforementioned long felt and unresolved needs of humanity, while being an economical production source for H₂, proteins and hydrocarbons. The instant invention surprisingly may be managed to produce: an algal protein product for food production, most preferably in animal feed; hydrocarbons, from which hydrocarbon fuels may be obtained; fertilizer; and many biochemical products. Therefore, the instant invention is more than a solution to long felt and unresolved environmental needs and needs to manage an ABR, the instant invention is economically practical from a business perspective; as, the instant invention produces marketable products for which there are defined market needs. This surprising economical combination of business/marketing practicality, along with the unexpected ability to meet the aforementioned long felt and unresolved human needs, is an aspect of the novelty and non-obviousness of the instant invention, which will further implementation of the instant invention.

### SUMMARY OF THE INVENTION

A primary object of the instant invention is to devise environmentally friendly, effective, efficient and economically feasible methods, processes and apparatus, wherein CO_{X} is sequestered.

Another object of the instant invention is to devise environmentally friendly, effective, efficient and economically feasible methods, processes and apparatus, wherein CO_{X} and/or NO_{X} from the combustion of a hydrocarbon is effectively and efficiently removed from a combustion exhaust.

Another object of the instant invention is to devise environmentally friendly, effective, efficient and economically feasible methods, processes and apparatus, wherein CO_{X} and/or NO_{X} from the combustion of a hydrocarbon is effectively and efficiently converted into a harmless salt.

Further, an object also of the instant invention is to devise environmentally friendly, effective, efficient and economically feasible methods, processes and apparatus, wherein CO_{X} and/or NO_{X} from the combustion of a hydrocarbon is effectively and efficiently converted into a harmless salt which can be easily disposed.

Still further, an object of the instant invention is to devise environmentally friendly, effective, efficient and economically feasible methods, processes and apparatus, wherein CO_{X} and/or NO_{X} from the combustion of a hydrocarbon is effectively and efficiently converted into a salt which has use as a soil stabilizer.

Still further yet, an object of the instant invention is to devise environmentally friendly, effective, efficient and economically feasible methods, processes and apparatus, wherein CO_{X} and/or NO_{X} from the combustion of a hydrocarbon are effectively and efficiently converted into a salt which has use as a building material.

Still further yet, an object of the instant invention is to devise environmentally friendly, effective, efficient and economically feasible methods, processes and apparatus, wherein CO_{X} and/or NO_{X} from the combustion of a hydrocarbon are effectively and efficiently converted into a salt which has use as a buffer of pH.

Still also further yet also, an object of the instant invention is to devise environmentally friendly, effective, efficient and economically feasible methods, processes and apparatus, wherein CO_{X} and/or NO_{X} from the combustion of a hydrocarbon are effectively and efficiently converted into a salt which can be reacted with an acid to release CO₂ and/or NO₂.

Further yet still, an object of the instant invention is to devise environmentally friendly, effective, efficient and economically feasible methods, processes and apparatus, wherein CO_{X} is converted into plant matter and O₂.

Further yet still also, an object of the instant invention is to devise environmentally friendly, effective, efficient and economically feasible methods, processes and apparatus, wherein NO_{X} from the combustion of a hydrocarbon is effectively and efficiently converted into N₂.

An object of the instant invention is to devise environmentally friendly, effective, efficient and economically feasible means, wherein CO_{X} is converted to O₂.

A secondary object of the instant invention is to devise environmentally friendly, effective, efficient and economically feasible means, wherein NO_{X} is converted to N₂.

A tertiary object of the instant invention is to devise environmentally friendly, effective, efficient and economically feasible means, wherein sulfides and oxides of sulfur are converted to elemental sulfur.

Another object of the instant invention is to devise environmentally friendly, effective, efficient and economically feasible means, wherein CO_{X} and/or NO_{X} and/or S_{X} from the combustion of a hydrocarbon is effectively and efficiently removed from combustion exhaust.

Further, an object of the instant invention is to devise environmentally friendly, effective, efficient and economically feasible means of an ABR, wherein energy is managed.

Further still, an object of the instant invention is to devise environmentally friendly, effective, efficient and economically feasible means of an ABR, wherein photon (light) contact with algae is managed.

Further yet, an object of the instant invention is to devise environmentally friendly, effective, efficient and economically feasible means of an ABR, wherein photon (light) is created from ABR hydrocarbon product so as to provide photons to the ABR.

Further still yet, an object of the instant invention is to devise environmentally friendly, effective, efficient and economically feasible means of an ABR, wherein the ABR produces O₂ and/or H₂.

Further yet still, an object of the instant invention is to devise an environmentally friendly, effective, efficient and economically feasible ABR Means, wherein required equipment and space are minimized.

Still further also yet, an object of the instant invention is to devise environmentally friendly, effective, efficient and economically feasible means of an ABR, wherein the products of the ABR have market potential, most preferably proteins and/or hydrocarbons so that the ABR has business/market potential, as well as ability to meet a long felt need of humanity.

Additional objects and advantages of the instant invention will be set forth in part in a description which follows and in part will be obvious from the description, or may be learned by practice of the invention.

The instant invention embodies incorporating CO_{X} and NO_{X} into an aqueous phase. The instant invention embodies the water adsorption characteristics of CO_{X} and/or NO_{X}. The instant invention further embodies combining at least one of CO_{X} and NO_{X} into metal salt(s), preferably into a Group IA or Group IIA metal salt, most preferably into a salt comprising at least one of sodium, magnesium or calcium. The instant invention further also embodies the affinity that a metal, preferably a Group IA metal or Group IIA metal, and most preferably at least one of sodium, magnesium or calcium, has for carbonate anions. The instant invention also further embodies the insolubility characteristics of a metal, preferably a Group IA IIA metal, most preferably at least one of sodium or calcium with carbonate, whether as a hydrate or in an anhydrous form. The instant invention further still embodies the anti-agglomeration characteristics of a dispersant in combination with a metal-CO₃ or a metal-NO₂ or a metal-NO₃ in aqueous solution.

The instant invention has surprisingly been discovered to inexpensively and safely remove at least one of CO_{X} and/or NO_{X} from a gas. In a most preferred embodiment, at least a portion of the CO_{X} and/or NO_{X} are adsorbed into an aqueous phase, wherein at least a portion of the CO_{X} and/or NO_{X} is reacted with a metal salt. It is preferred that the metal salt be added to the aqueous phase as at least one selected from the group consisting of: calcium sulfate, calcium sulfate ½ hydrate, calcium sulfate hydrate, calcium sulfate di-hydrate, and any combination therein.

This instant invention is surprisingly found to be easily configured in a variety of process and equipment arrangements such that the instant invention can be easily added to any source of CO_{X} and/or NO_{X}. The instant invention is surprisingly found to be practically added to modes of transportation, e.g. a motorcycle, an automobile, a truck, a boat, or etc. The instant invention has surprisingly been found to practically be added to the exhaust stack of a power plant, a manufacturing plant, a furnace or any type of combustion method, process or device. The instant invention has surprisingly been found to be economically practical in application and in use, wherein economics and practicality are important characteristics of an invention such as the instant invention which has to have broad appeal in order to be implemented. Finally, the instant invention has surprisingly been found to be an economical and practical means to store CO_{X} and/or NO_{X} be that above or below ground. This instant invention is surprisingly found to be easily configured in a variety of process and equipment arrangements such that the instant invention can be easily added to any source comprising CO_{X}. The instant invention has surprisingly been found to practically be added to the exhaust stack of a power plant, a manufacturing plant, a furnace or any type of hydrocarbon combustion means or hydrocarbon source comprising CO_{X}. The instant invention has surprisingly been found to be economically practical in application and in use, wherein economics and practicality are important characteristics of an invention such as the instant invention which has to have broad appeal in order to be implemented on the scale needed by humanity.

### BRIEF DESCRIPTION OF THE DRAWINGS

A better understanding of the instant invention can be obtained when the following descriptions of the preferred embodiments are considered in conjunction with the following drawings, in which:

FIGS. 1 and 1.1 illustrate a legend for FIGS. 2 through 17.

FIG. 2 illustrates a graphical representation of a Gas Scrubber [1] to adsorb/precipitate available Gas Flow into an aqueous phase in combination with an optional Salt Reactor [2] to convert any remaining CO_{X} and/or NO_{X} into a final metal salt, wherein a Separator [3] separates precipitated final metal salt(s) from the aqueous phase.

FIG. 3 illustrates a graphical representation of a Gas Scrubber [1] to adsorb/precipitate available CO_{X} and/or NO_{X} into an aqueous phase in combination with an optional Salt Reactor [2] to convert the available CO_{X} and/or NO_{X} into a final metal salt, wherein a Separator [3] separates precipitated final metal salt(s) from the aqueous phase, wherein the aqueous phase is recycled back to the Gas Scrubber [1], wherein further adsorption/precipitation occurs in a Salt Reactor [2A] in combination with further separation in Separator [3A], and wherein the aqueous phase is recycled to the Gas Scrubber [1] for further adsorption/precipitation of available CO_{X} and/or NO_{X} into aqueous phase.

FIG. 4 illustrates a graphical representation of a Gas Scrubber [1] to adsorb/precipitate available CO_{X} and/or NO_{X} into an aqueous phase in combination with an optional Salt Reactor [2] to convert the available CO_{X} and/or NO_{X} into a final metal salt, wherein a Separator [3] separates precipitated metal salt(s) from the aqueous phase, wherein a Greenhouse [4] converts the precipitated CO₃²⁻ back into CO₂ for conversion into O₂ with algae, wherein a Separator [5] separates final metal salt(s) from the wastewater, and wherein said algae is available for harvesting.

FIG. 5 illustrates a graphical representation of a Gas Scrubber [1] to adsorb/precipitate available CO_{X} and/or NO_{X} into an aqueous phase in combination with an optional Salt Reactor [2] to convert the available CO_{X} and/or NO_{X} into a final metal salt, wherein a Separator [3] separates precipitated final metal salt(s) from the aqueous phase, wherein a Greenhouse [4] converts the precipitated CO₃²⁻ back into CO₂ for conversion into O₂ with algae, wherein a Separator [5] separates precipitated final metal salt(s) from the wastewater, wherein an Facultative Bio-Reactor [6] converts NO₂²⁻ and NO₃²⁻ within the wastewater into N₂, wherein a Separator [7] separates the wastewater from the bio-solids of the Facultative Bio-Reactor [6], and wherein said algae is available for harvesting.

FIG. 6 illustrates a graphical representation of a Catalysis Unit [8] to convert at least a portion of any NO_{X} combustion gases into N₂, along with a downstream Gas Scrubber [1] to adsorb/precipitate available CO_{X} and/or NO_{X} into an aqueous phase, in combination with an optional Salt Reactor [2] to convert any remaining CO_{X} and/or NO_{X} into a final metal salt, wherein a Separator [3] separates precipitated final metal salt(s) from the water phase.

FIG. 7 illustrates a graphical representation of a Catalysis Unit [8] to convert at least a portion of any NO_{X} combustion gases into N₂, along with a downstream Gas Scrubber [1] to adsorb/precipitate available CO_{X} and/or NO_{X} into an aqueous phase, in combination with an optional Salt Reactor [2] to convert the available CO_{X} and/or NO_{X} into a final metal salt, wherein a Separator [3] separates precipitated final metal salt(s) from the aqueous phase, wherein the aqueous phase is recycled back to the Gas Scrubber [1], wherein further adsorption/precipitation occurs in a Salt Reactor [2A] in combination with further separation in Separator [3A], and wherein the aqueous phase is recycled to the Gas Scrubber [1] for further adsorption/precipitation of available CO_{X} and/or NO_{X} into aqueous phase.

FIG. 8 illustrates a graphical representation of a Catalysis Unit [8] to convert at least a portion of any NO_{X} combustion gases into N₂, along with a downstream Gas Scrubber [1] to adsorb/precipitate available CO_{X} and/or NO_{X} into an aqueous phase, in combination with an optional Salt Reactor [2] to convert the available CO_{X} and/or NO_{X} into a final metal salt, wherein a Separator [3] separates precipitated metal salt(s) from the aqueous phase, wherein a Greenhouse [4] converts the precipitated CO₃²⁻ back into CO₂ for conversion into O₂ with algae, wherein a Separator [5] separates precipitated metal salt(s) from the wastewater, wherein an Facultative Bio-Reactor [6] converts NO₂²⁻ and NO₃²⁻ within the wastewater into N₂, wherein a Separator [7] separates the wastewater from the bio-solids of the Facultative Bio-Reactor [6], and wherein said algae is available for harvesting.

FIG. 9 illustrates a graphical representation of a Gas Scrubber [1] to adsorb/precipitate available CO_{X} and/or NO_{X} from a Gas flow into an aqueous solution. The aqueous solution from the Scrubber flows to ABR(s) [9], wherein CO_{X} and/or NO_{X} are converted into biomass (biomass is herein defined as comprising at least one of algae and bacteria) and O₂. The final H₂ or O₂ product is separated from ABR aqueous solution effluent by means a separator, which is preferably of cyclone design [3]. Aqueous solution comprising algae is wasted from the ABR(s) Recycle Loop, after which the algae is at least partially separated from ABR aqueous solution with a Separator [7], which can be a centrifuge, clarifier, filter, or any similar liquids/solids separation device as is known in the art of liquids/solids separation.

FIG. 10 illustrates a graphical representation of a Gas flow to Tubular ABR(s) [9], wherein Gas flow comprising CO_{X} and/or NO_{X} are converted into biomass and O₂. It is understood that said Tubular ABR(s) may be replaced with any ABR design of the instant invention, e.g. Cluster(s), Continuous Stirred Tank Rectors (CSTR(s)), etc. ABR aqueous solution is separated into a gas and a liquid effluent by means a separator, which is preferably of cyclone design [3]. Liquid comprising algae is wasted, after which the algae is at least partially separated from the liquid with a Separator [7], which can be a centrifuge, clarifier, filter, or any similar liquids/solids separation means as is known in the art. Algae is harvested by dewatering wasted algae from Liquids/Solids Dewatering Equipment [7A], which can be a centrifuge, belt filter press, filter press, or any similar dewatering liquids/solids separation means for dewatering. When sulfur removal is performed via Facultative Biological Reactor (FBR) [6], FBR liquid effluent is to be separated, wherein the case of the FBR solids dewatering, sulfur is separated from the biological mass. O₂ generated in the ABR is separated from ABR(s) gaseous effluent in separator [3C], which can be one of cryogenic distillation, membrane separation, and pressure or vacuum swing adsorption. Optionally, FBR [6] converts any NO into N₂ and/or any S_{X} into S. A light collection system [10], preferably with ability to track location of the Sun and orient the collection system for optimal effectiveness in orientation to the Sun, gathers photons, which are transferred to the ABR(s). Photon distribution point [10A], which is preferably spherical in shape with a mirrored surface on the interior, nearly evenly distributes photons to each ABR(s).

FIG. 11 illustrates a graphical representation of Gas flow to ABR(s) [9] and ABR(s) [9A], wherein CO_{X} and/or NO_{X} are converted into biomass, O₂ and H₂. It is understood that said Tubular ABR(s) may be replaced with any ABR design of the instant invention, e.g. Cluster(s), CSTR(s), etc. As the hydrogenase algal reaction producing H₂ requires regeneration by O₂ production, it is preferred that at least one ABR produce O₂ while at least one ABR(s) produce H₂, after which the H₂ producing algae can be regenerated in the O₂ producing ABR(s) (this is best be performed with three ABR(s), wherein two at a time are producing O₂ and one at a time is producing H₂). The final ABR(s) gaseous product is separated from ABR(s) aqueous solution effluent by means a separator, which is preferably of cyclone design [3] and [3A]. Liquid comprising algae is wasted, after which the algae is at least partially separated from the liquid with Separation Equipment [7] and [7A], which can be a centrifuge, clarifier, filter, or any similar liquids/solids separation means as is known in the art. Algae is then dewatered with Separation Equipment [7C], which can be a centrifuge, belt filter press, filter press, or any similar dewatering liquids/solids separation means for dewatering. O₂ generated in the ABR(s) is separated from ABR(s) gaseous effluent in separator [3C], which can be one of: cryogenic distillation, membrane separation, and pressure or vacuum swing adsorption. H₂ generated in the ABR(s) is separated from ABR(s) gaseous effluent in separator [3D], which can be one of: cryogenic distillation, membrane separation, and pressure or vacuum swing adsorption. Optionally, FBR [6] converts any NO_{X} into N₂ and/or any S_{X} into S. FBR [6A] converts any NO_{X} into N₂ and/or any S_{X} into S, thereby a means of S reduction in the H, producing ABR(s). When sulfur removal is performed via FBR [6] or FBR [6A], wasted FBR liquid effluent is to be separated by means similar to that of algae separation and dewatering, wherein the case of the FBR solids dewatering, sulfur is separated from the biological mass. A light collection system [8], preferably with ability to track location of the Sun and orient the collection system for optimal effectiveness in orientation to the Sun, gathers photons, which are transferred to the ABR(s). Photon distribution point [8A], which is preferably spherical in shape with a mirrored surface on the interior, nearly evenly distributes photons to each ABR(s).

FIG. 12 illustrates a graphical representation of a single tubular ABR. While a single ABR is depicted in FIG. 12, as well as in each ABR(s) depiction in FIGS. 9, 10 and 11, it is to be understood that each ABR depiction may represent numerous ABR(s), an ABR Cluster as taught herein, a CSTR ABR, numerous ABR Cluster, or numerous CSTR ABR as taught herein.

FIG. 13 illustrates a graphical representation of the most preferred ABR Cluster means.

FIG. 14 illustrates a graphical representation of the flow schematic for an ABR Cluster, along with an ABR Cluster means which is an embodiment, while not the preferred embodiment, of the instant invention.

While FIGS. 13 and 14 depict ABR Cluster, wherein the ABR are adjacent to each other, it is an embodiment as depicted in FIG. 8 which illustrates a graphical representation of the ABR(s) such that photons from the photon tube may pass between the ABR(s), wherein the photons which pass between the ABR(s) may be reflected from a reflective or mirrored surface behind the ABR(s) and onto the portion (backside) of the ABR(s) which does not face the photon tube.

FIG. 15 illustrates a graphical representation of an embodiment comprising a number of ABR, wherein a photon tube is located between each ABR.

FIG. 16 illustrates a CSTR ABR with photon tubes, gas tubes, a mirrored outside surface surrounded by insulation.

FIG. 17 illustrates an ABR Cluster in an annular arrangement comprising photon tubes, gas tubes, a mirrored outside surface surrounded by insulation.

### DETAILED DESCRIPTION OF THE PREFERRED EMBODIMENTS

Timing of the instant invention is significant and meets a long felt need as global climate change is changing weather patterns around the Earth. Timing of the instant invention is significant and meets a long felt need as global climate change is becoming a global political issue. Timing of the instant invention is significant and meets a long felt need since the products of hydrocarbon combustion are now affecting the health of humanity, as well as that of animals, plant and sea life on Earth.

The instant invention is described in connection with one or more preferred embodiments. However, it should be understood that the invention is not limited to those embodiments. In contrast, the invention includes all alternatives, modifications and equivalents as may be included within the split and scope of the specification and of the appended claims.

The instant invention provides means for the sequestration and/or conversion of Gas comprising CO_{X}, as well as comprising at least one of, NO and S_{X} (Gas is herein defined as comprising at least one of CO_{X} and NO_{X}, and may comprise S. The instant invention embodies means of converting a Gas into at least one of a salt and biomass. In the case of biomass, conversion further comprises converting into O₂ and potentially H₂. The salt conversion means comprises contacting the gas with water, therein forming an aqueous solution, wherein the water comprises a metal salt, such that in the water is formed a final metal salt in aqueous solution, wherein the final metal salt in aqueous solution comprises the metal and CO₃, and wherein the aqueous solution comprises a dispersant. The biomass means comprises: 1) contacting the Gas with water, therein forming an aqueous solution, or 2) contacting the Gas with water, therein forming an aqueous solution, wherein the water comprises a metal salt, such that in the water is formed a final metal salt in aqueous solution, and wherein the final metal salt in aqueous solution comprises the metal and CO₃, and optionally 3) contacting the Gas with water, therein forming an aqueous solution, wherein the water comprises a metal salt, such that in the water is formed a final metal salt in aqueous solution, wherein the final metal salt in aqueous solution comprises the metal and CO₃, and wherein the aqueous solution comprises a dispersant. Aqueous solution 1 or 2 or 3 is formed prior to contacting with algae in at least one ABR, wherein the ABR converts into biomass at least a portion of at least one of: the CO_{X}, metal CO₃ salt, NO_{X}, metal NO₃ salt, and any combination therein. The instant invention further embodies when the ABR converts into biomass and/or N₂ gas at least a portion of at least one of the NO_{X}, NO₂ and NO₃. It is preferred that the Gas is from a combustion source or a source of hydrocarbon(s). It is preferred that the gas conversion produce O₂. It is preferred that the gas comprise Gas Flow.

The instant invention embodies the adsorption of at least one CO_{X} and/or NO molecule into an aqueous phase, thereby creating an aqueous phase comprising the CO_{X} and/or NO_{X} molecule(s). The instant invention embodies the adsorption of at least one CO_{X} and/or NO molecule from a hydrocarbon combustion source into an aqueous phase, thereby creating an aqueous phase comprising said CO_{X} and/or NO_{X} molecule(s). The instant invention further embodies the reaction of said aqueous phase CO_{X} and/or NO_{X} molecule(s) with a metal to further form an aqueous salt solution comprising the metal and a CO₃ and/or NO_{2 or 3} molecule(s). The instant invention further embodies the reaction of said aqueous phase molecule(s) with a Group IA and/or IIA metal to further form an aqueous salt solution comprising the Group IA and/or IIA metal and the CO₃ and/or NO_{2 or 3} molecule(s). The instant invention further still embodies the reaction of said aqueous salt solution with a metal to a point wherein said salt in said aqueous salt solution is at a concentration beyond its solubility point, such that the metal salt precipitates from said aqueous salt solution. It is most preferred that said metal salt comprise a Group IA metal for the formation of an insoluble salt comprising CO₃. It is most preferred that said metal salt comprise at least one of sodium or calcium for the formation of an insoluble salt comprising CO₃. It is most preferred that said metal salt comprise iron or magnesium for the formation of an insoluble salt comprising CO₃. It is most preferred that said Group IA and/or HA metal salt comprise a Group IA metal for the formation of a insoluble salt comprising NO_{2 or 3}. It is most preferred that said metal salt comprise potassium for the formation of an insoluble salt comprising NO_{2 or 3}. It is an embodiment that the Group IA and/or IIA metal is replaced with at least one element selected from the group consisting of a: IIIA, IVA, IB, IIB, IIIB, IVB, VB, VIIB, VIIB, VIIIB and any combination therein.

### Chemical Equilibria

Chemical Equilibria of the instant invention include but are not limited to:

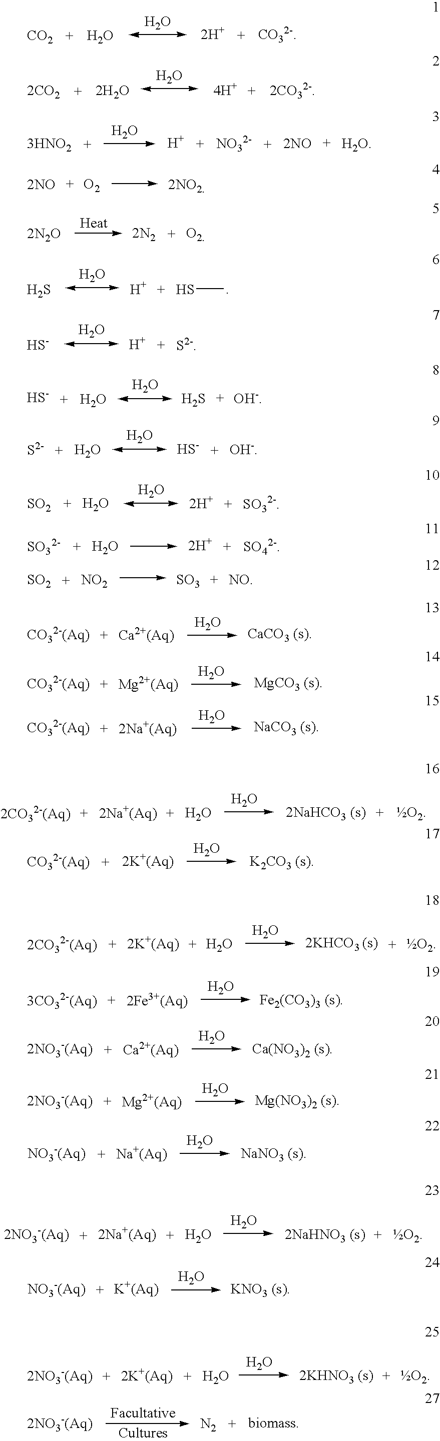

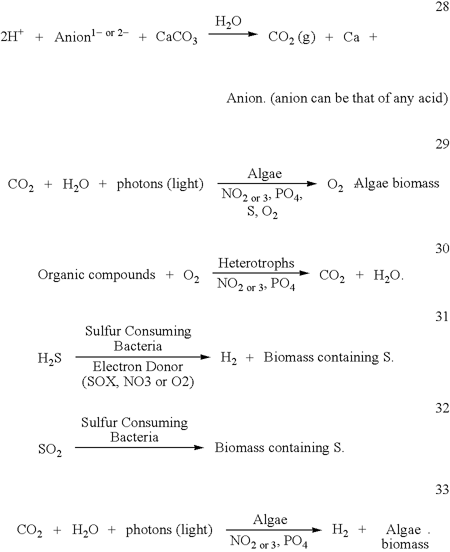

The instant invention embodies the addition of a dispersant to the aqueous solution comprising the metal salt precipitate(s). The instant invention embodies the addition of a dispersant to the aqueous solution such that the addition of the dispersant allows for further aqueous adsorption of CO_{X} and/or NO_{X} molecule(s) into the aqueous phase. This further aqueous phase adsorption is preferably performed without an agglomeration of the metal salt precipitate(s) inhibiting further aqueous phase adsorption of CO_{X} and/or NO_{X} molecule(s).

It is an embodiment that the metal be added to the aqueous solution in the form of a salt. It is preferred that the metal for the formation of an insoluble salt comprising CO₃ comprise at least one selected from the group consisting of: sodium sulfate (Na₂SO₄), sodium sulfate heptahydrate (Na₂SO₄.7H₂O), sodium sulfate decahydrate (Na₂SO₄.10H₂O), sodium bisulfate (NaHSO₄), sodium

### Water Solubility Relationships

TABLE 1
Solubility in H₂O¹

| | (mg/100 ml H₂O)² | | | (mg/100 ml H₂O)² | |
|---|---|---|---|---|---|
| Gas | Cold H₂O | Hot H₂O | Gas | Cold H₂O | Hot H₂O |
| CO | 3.5 | 2.3 | H₂S | 437 cm³ | 186 cm³ |
| CO₂ | 0.348 | 0.097 | SO₂ | 22.8 | 0.58 |
| CO₃ | Soluble | Soluble | SO₃ | Decomposes | Decomposes |
| | | | | to H₂SO₄ | to H₂SO₄ |
| NO | 7.34 cm³ | 2.37 cm³ | SO₄²⁻ | Forms | Forms H₂SO₄ |
| | | | | H₂SO₄ or a | or a metal salt |
| | | | | metal salt | |
| N₂O | 130.0 | 56.7 | | | |
| NO₂ | Soluble | Decomposes | | | |
| NO₃ | Soluble | Soluble | | | |

| Metal | Anion CO₃ (mg/100 ml H₂O)² | | | | Anion NO₃ (mg/100 ml H₂O)² | | | |
|---|---|---|---|---|---|---|---|---|
| Cation | Cold H₂O | | Hot H₂O | | Cold H₂O | | Hot H₂O | |
| Ca | | 0.0015 | | 0.0019 | | 121.2 | | 376.0 |
| Mg | | 0.0106 | | — | | Soluble | | Soluble |
| Na | | 7.1000 | | 45.5000 | | 92.1 | | 180.0 |
| K | | 112.0000 | | 156.0000 | | 7.0 | | 60.8 |
| Fe | II | Insoluble | II | Insoluble | II | 83.5 | II | 156.7 |
| | III | Insoluble | III | Insoluble | III | Soluble | III | Soluble |
| Mn | | 0.0065 | | Insoluble | | 456.4 | | Soluble |

| | Anion HSO₄ | | Anion SO₄ | |
|---|---|---|---|---|
| | (mg/100 ml H₂O)² | | (mg/100 ml H₂O)² | |
| Metal Cation | Cold H₂O | Hot H₂O | Cold H₂O | Hot H₂O |
| Ca | Soluble | Soluble | 0.209 | 0.162 |
| Mg | Soluble | Soluble | 20.0 | 73.8 |
| Na | Soluble | Soluble | 4.76 | 42.7 |
| K | 36.3 | 121.6 | 12.0 | 24.1 |

¹Reference CRC Handbook of Chemistry and Physics, 56'th Edition, CRC Press, 1975
²Unless otherwise noted.

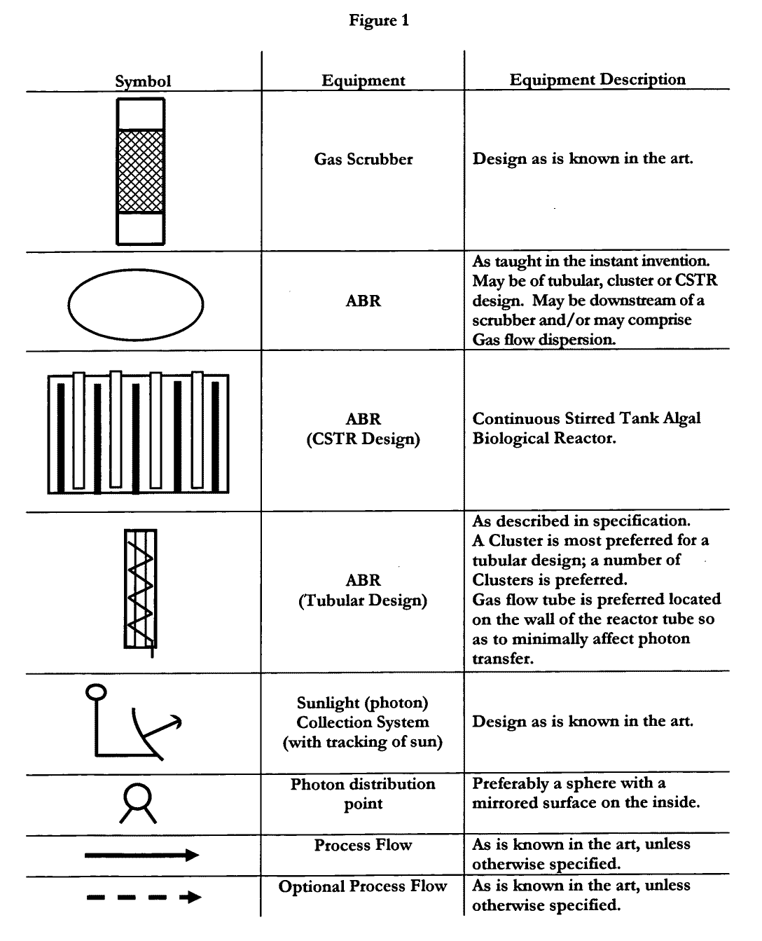

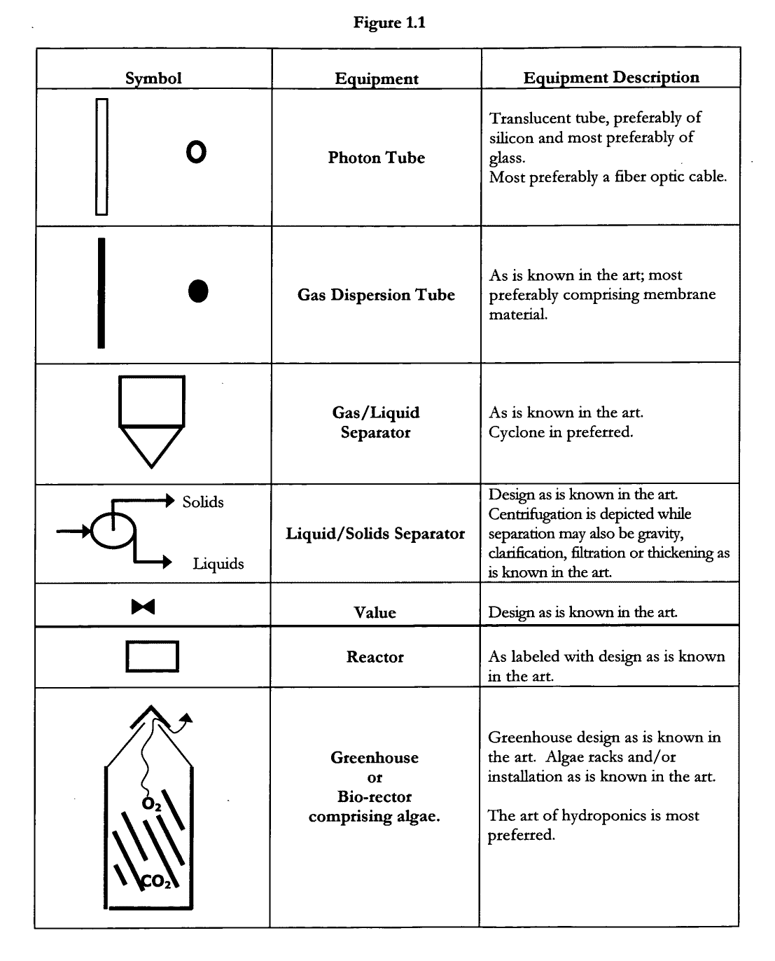

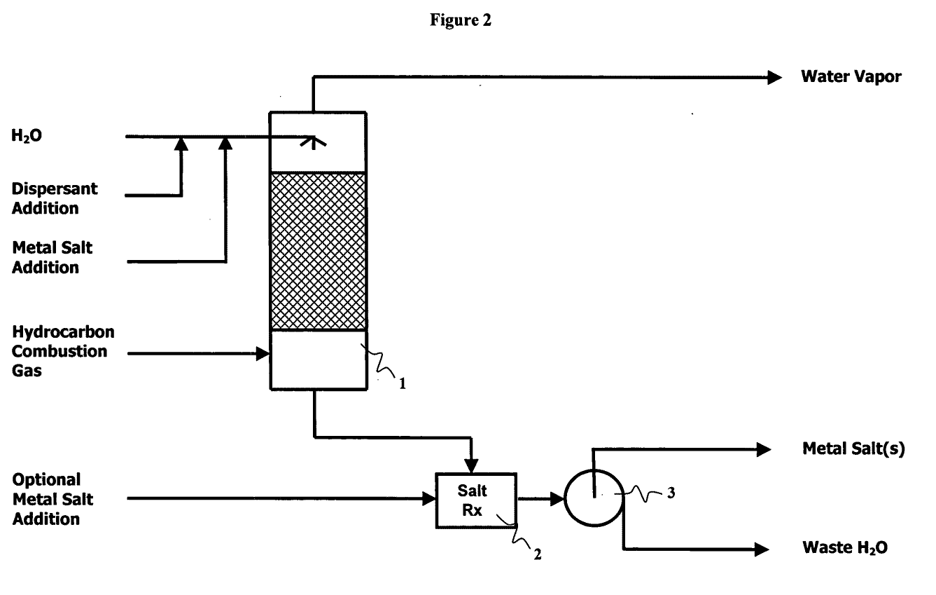

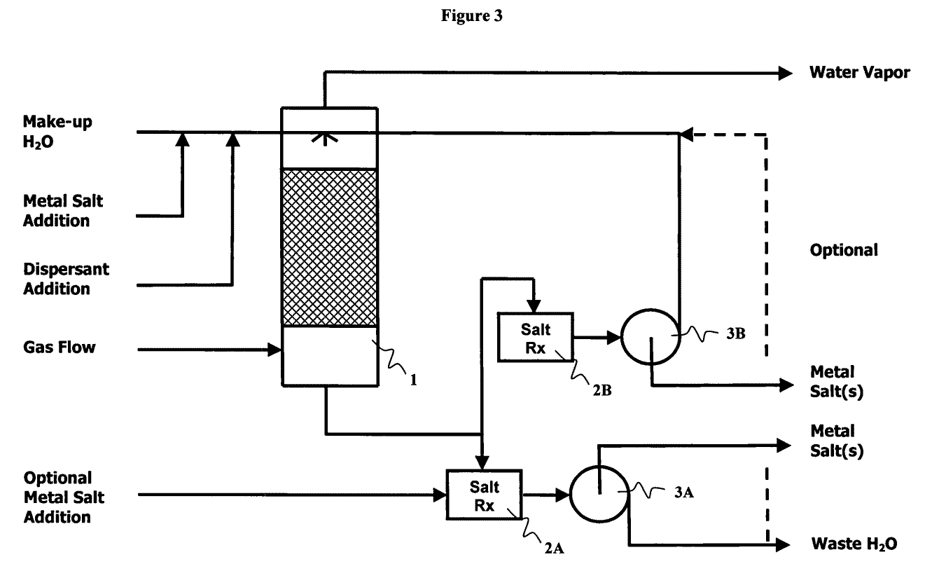

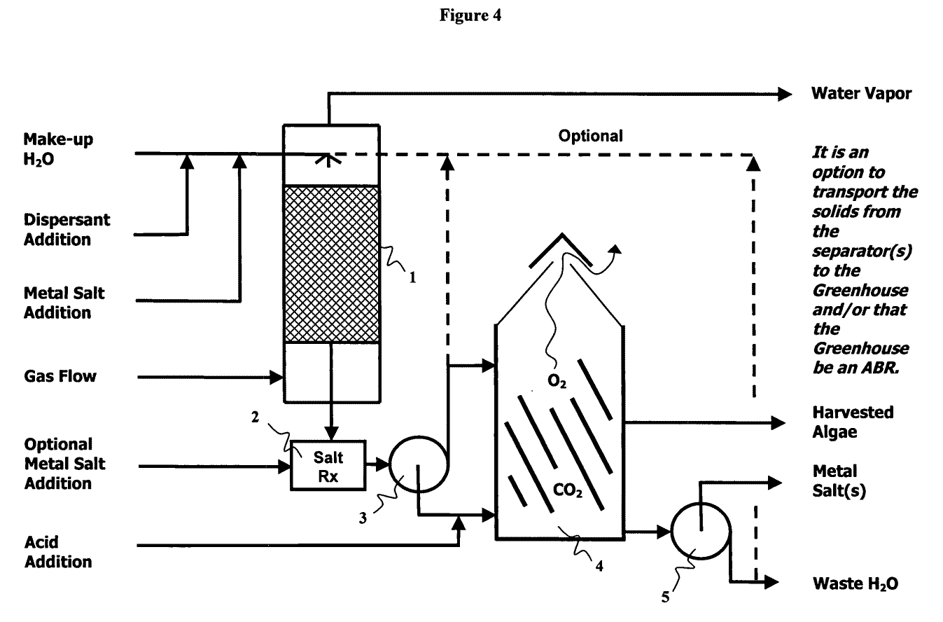

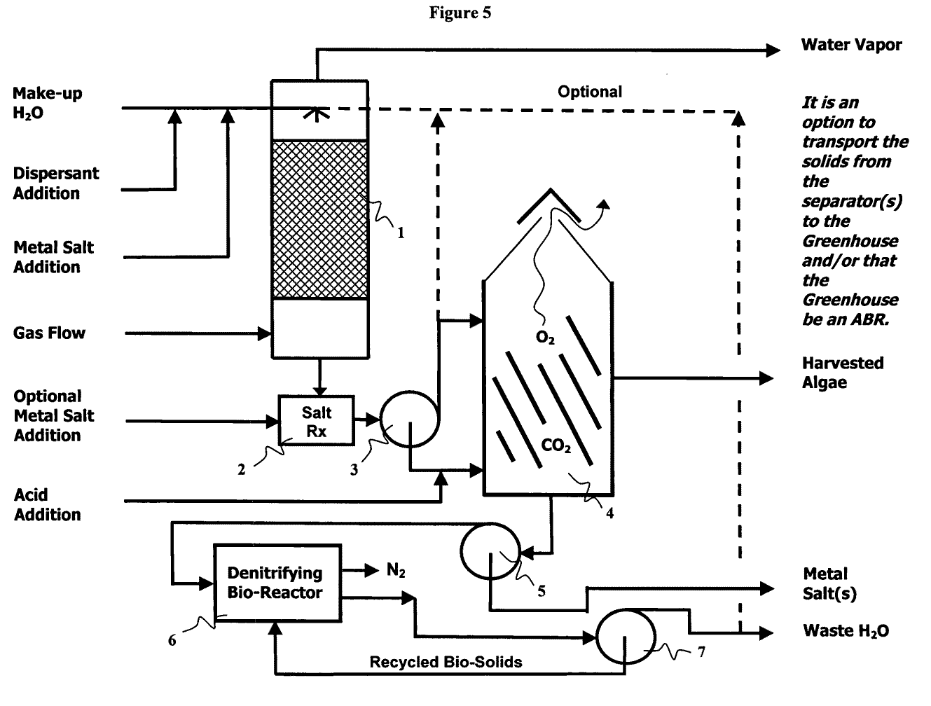

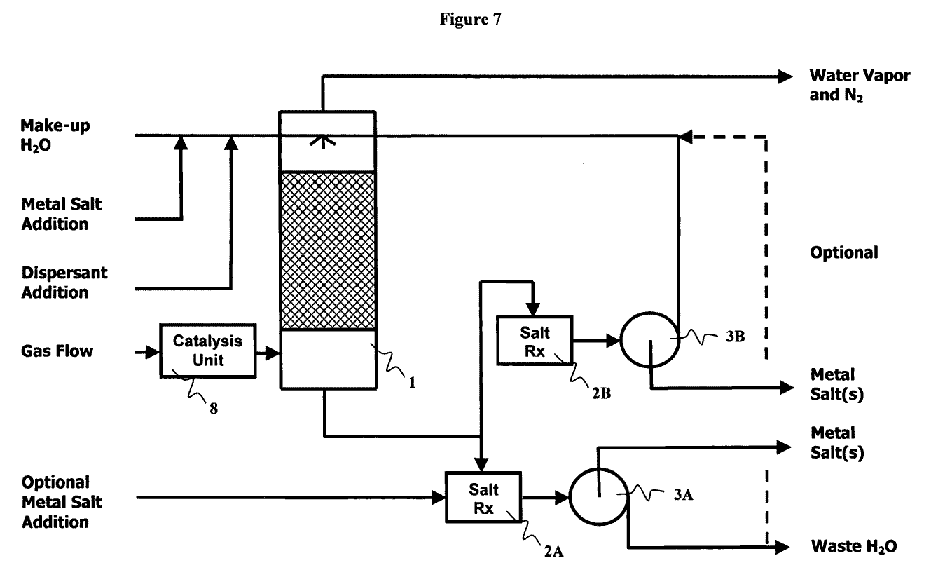

bisulfate monohydrate (NaHSO₄.H₂O), calcium sulfate (CaSO₄), calcium sulfate ½ hydrate (CaSO₄.½H₂O), calcium sulfate hydrate (CaSO₄.H₂O), calcium sulfate di-hydrate (CaSO₄.2H₂O), potassium sulfate (K₂SO₄), potassium bisulfate (KHSO₄), potassium sulfate ½ hydrate (K₂SO₄.½H₂O), potassium sulfate hydrate (K₂SO₄.H₂O), potassium sulfate di-hydrate (K₂SO₄.2H₂O), and any combination therein. It is preferred that the metal for the formation of an insoluble salt comprising NO_{X} comprise at least one selected from the group consisting of potassium sulfate (K₂SO₄), potassium sulfate ½ hydrate (K₂SO₄. ½H₂O), potassium sulfate hydrate (K₂SO₄.H₂O), potassium sulfate di-hydrate (K₂SO₄.2H₂O), and any combination therein. It is most preferred that the metal salt comprise a base so as to keep the metal solution alkaline. It is most preferred that the base comprise at least one of: sodium, potassium, calcium and magnesium. It is most preferred that the base comprise at least one of hydroxyl and oxygen anionic moiety.
Scrubber—It is an embodiment to have a gas/water contact device (herein defined as a Scrubber) to contact a gas comprising CO_{X} and preferably comprising at least one of NO_{X} and S_{X} (Gas flow) with H₂O in order to create a solution comprising CO_{X} and/or NO_{X} and/or S_{X}. It is preferred that the Scrubber be of vertical type as is known in the art or as depicted in FIGS. 1 and 2 through 9. It is preferred that the temperature of the gas or water entering the scrubber be greater than about 45° C. so as to limit mesophilic biological growth in the scrubber. It is most preferred that the Gas flow or water entering the Scrubber be greater than about 70° C. It is preferred that the Scrubber be greater than about 45° C. so as to limit mesophilic biological growth in the scrubber. It is most preferred that the Scrubber be greater than about 70° C. so as to limit mesophilic and thermophilic biological growth in the Scrubber. It is preferred that the water entering the Scrubber comprise a dispersant. It is preferred that the water entering the Scrubber comprise a metal salt so as to facilitate the formation of the corresponding metal CO₃ or NO_{2 or 3} salt in aqueous solution. It is an embodiment that the Scrubber comprises metal construction. It is preferred that the Scrubber comprises a material which is capable of structural integrity at exhaust gas temperatures available from hydrocarbon combustion or operating Scrubber temperatures. It is preferred that the Scrubber comprises at least one selected from the group consisting of zirconium, hastelloy, titanium and inconnel, or corrosion resistant metals of the like; polynylon, polyester (PET or PBT), polyetherimide, polyimide, polypropylene, or polymers of the like; glass; and any combination therein. It is preferred that downstream of the Scrubber be a cooler which cools Scrubber exit aqueous solution prior to entrance of the Scrubber exit aqueous solution into an ABR. It is preferred that upstream of the Scrubber be a cooler which cools Scrubber inlet water prior to entrance of the Scrubber. It is preferred that the Scrubber comprise a packing material so as to facilitate contact between the Gas and the aqueous phase in the scrubber.

Further, to the extent that a 3-way catalytic converter is not converting NO_{X} to N₂, the aqueous phase in a scrubber can hold about; 120 to 370 gm of Ca(NO₃)₂ per 100 cc of H₂O depending on temperature, or 125 gm or greater of Mg(NO₃)₂ per 100 cc of H₂O depending on temperature, or 92 to 180 gm of NaNO₃ per 100 cc of H₂O depending on temperature, or 13 to 247 gm of KNO₃ per 100 cc of H₂O, depending on temperature; wherein any concentration beyond the solubility limit will precipitate as the corresponding metal-NO₃ salt. The adsorption of NO₃⁻ in the aqueous phase and the corresponding metal-NO₃ salt has two advantages: first, NO_{X} emissions are at least partially controlled; and second, there is a ready measure of catalytic converter performance, e.g. conversion of NO_{X} to N₂, as any concentration of NO₂ or of NO₃ in the aqueous phase and/or salt in comparison to fuel use is a direct measure of catalytic converter NO_{X} performance. It is anticipated for catalytic converter maintenance to be more economical than the removal of NO₂⁻ or of NO₃⁻ from either the aqueous solution (phase) or the precipitate.

It is a most preferred embodiment to operate the Scrubber wherein at least one of CO₂ and NO₃ metal salt precipitation is performed, and wherein the dispersant is added to the Scrubber to reduce precipitation formation on surfaces. It has surprisingly been found that operating the Scrubber with the metal salt precipitation allows for the Scrubber to be significantly more effective and efficient due to the amount of CO₃ and/or NO₃ placed in solution via metal salt chemistry as compared to that placed in solution via CO₃ and/or NO₃ solubility, as can be seen in Table 1.

It is an embodiment to locate the Scrubber in the exhaust piping of a combustion device or engine, wherein the Scrubber has the means to adsorb at least a portion of the CO_{X} and/or NO_{X} produced in combustion. It is preferred that the Scrubber be sized so as to allow for at least a portion of the CO_{X} and/or NO_{X} produced in combustion to be adsorbed in the Scrubber aqueous phase. It is most preferred that the Scrubber be sized so as to allow for at about most to all of the CO_{X} and/or NO_{X} produced in combustion to be adsorbed in the Scrubber aqueous phase. It is preferred that the water for the Scrubber comprise an acid or a disinfecting moiety so as to control or minimize precipitate and/or biological growth in the Scrubber. It is preferred that the concentration of dispersant in the Scrubber be maintained so as to afford the Scrubber means to adsorb most to all of the CO_{X} and/or NO_{X} produced in combustion in the aqueous phase without agglomeration or plugging of the Scrubber by an unmanageable amount of precipitate. It is preferred that the Scrubber have an easy method of water removal and addition. It is most preferred that the water reservoir for the Scrubber be sized so as to allow for most to about all of the CO_{X} and/or NO_{X} produced in combustion to be adsorbed in the aqueous phase, e.g. scrubber water, in the form of a soluble salt or in the form of a precipitate. It is most preferred that the Scrubber and Scrubber water reservoir have a means of energy management so that the composition of the water therein can be managed in relation to water vapor formation and water freezing.

Dispersion Water Chemistry—A dispersant is preferably added to the aqueous solution to prevent scale and/or precipitation on surfaces. Dispersants are low molecular weight polymers, usually organic acids having a molecular weight of less than 25,000 and preferably less than 10,000. Dispersant chemistry is preferably based upon carboxylic chemistry, as well as alkyl sulfate, alkyl sulfite and alkyl sulfide chemistry; it is the oxygen atom that creates the dispersion, wherein oxygen takes its form in the molecule as a carboxylic moiety and/or a sulfoxy moiety. Dispersants preferred which contain the carboxyl moiety include at least one selected from the group consisting of acrylic polymers, acrylic acid, polymers of acrylic acid, methacrylic acid, maleic acid, fumaric acid, itaconic acid, crotonic acid, cinnamic acid, vinyl benzoic acid, any polymers of these acids and any combination therein. Dispersants that can be used contain the alkyl sulfoxy or allyl sulfoxy moieties include any alkyl or allyl compound, which is water soluble containing a moiety that is at least one of SO, SO₂, SO₃, SO₄ and/or any combination therein. Due to the many ways in which an organic molecule can be designed to contain the carboxyl moiety and/or the sulfoxy moiety, it is an embodiment that any water soluble organic compound containing at least one of a carboxylic moiety and/or a sulfoxy moiety may be a dispersant in the instant invention. (This is with the knowledge that not all dispersants have equivalent dispersing properties.) Acrylic polymers exhibit very good dispersion properties, thereby limiting the deposition of water soluble salts and are most preferred embodiments as a dispersant. The limitation in the use of a dispersant is in the dispersants water solubility in combination with its carboxylic nature and/or sulfoxy nature.
Salt Reactor—It is preferred that said Salt Reactor(s) comprise an agitation of a metal salt so as to provide mixing of a metal salt with the aqueous solution from said Scrubber. It is preferred that the Salt Reactor(s) comprise an auger-type of design to provide mixing of the metal salt with the aqueous solution from said Scrubber. It is most preferred that the Salt Reactor(s) comprise a grinding devise so as to prevent the agglomeration of metal CO₃ and/or NO_{2 or 3} precipitate which could either affect Salt Reactor mixing of said metal salt with said aqueous solution from said Scrubber or affect the flow of said aqueous solution from said Scrubber through said Salt Reactor(s).

It is preferred that the Salt Reactor(s) comprise a means for adding fresh metal salt to the Salt Reactor(s). It is preferred that the Salt Reactor(s) comprise a means for removing solids from the Salt Reactor(s). It is most preferred that the Salt Reactor(s) operate with an excess of metal salt over that anticipated in the formation of the corresponding metal-CO₃ and/or metal-NO_{2 or 3}.

It is preferred to locate a Salt Reactor, wherein the exit water, aqueous phase, from said Scrubber enters the Salt Reactor, and wherein at least one of CO₃ and NO_{2 or 3} react with a metal salt in the Salt Reactor to form a metal-CO₃ and/or a metal-NO_{2 or 3} precipitate. It is preferred that the Salt Reactor be sized such that the Salt Reactor can convert at least a portion of the CO_{X} and/or NO_{X} in the aqueous phase from the Scrubber to a metal-CO₃ and/or a metal-NO_{2 or 3}. It is most preferred that the Salt Reactor and the water reservoir be sized such that the Salt Reactor can convert most to all of the CO_{X} and/or NO_{X} in the aqueous phase from the Scrubber to a metal-CO₃ and/or a metal-NO_{2 or 3}, wherein a portion of the CO_{X} in the aqueous phase precipitates as a metal-CO₃ and/or a portion of the NO_{2 or 3} precipitates as a metal-NO_{2 or 3} and wherein in aqueous solution is at least a portion of the remaining metal-CO₃ and/or metal-NO_{2 or 3}. It is preferred that the Salt Reactor comprises an easy means of removing at least one of: any unused metal salt and any metal-CO₃ and/or a metal-NO_{2 or 3} formed. It is preferred that the Salt Reactor have an easy means of fresh salt addition.

It is preferred that the metal salt in said Salt Reactor comprise at least one metal cation. It is most preferred that said metal cation comprise at least one selected from the group consisting of a metal, a Group IA or IIA metal, calcium, magnesium, sodium, potassium, a group VIII metal, iron, manganese, and any combination therein. It is preferred that the metal salt in said Salt Reactor comprises at least one anion selected from the group consisting of sulfate, sulfite, bisulfate, bisulfite, oxide, hydroxide, a halogen, chloride, bromide, nitrate, nitrite, hydride, and any combination therein. It is preferred that the metal salt in the salt reactor comprise an oxidizer capable of maintaining an alkaline pH in said Salt Reactor. It is most preferred that the pH in said Salt Reactor be between about 7.0 and about 10.0. It is an embodiment that the pH in said Salt Reactor be between about 6.0 and about 14.0.

Separator—It is an embodiment to locate a Separator downstream of said Scrubber and/or of said Salt Reactor so that the metal salts can be separated from aqueous solution. The Separator can be of any design as is known in the art. It is preferred that the separator be of gravity separation type of design, such as that which is known in a clarifier or in a thickener or in a belt dewatering press type of means. It is most preferred that the Separator be of centrifugation type of design.
Aqueous Recycle—It is an embodiment to recycle said aqueous salt solution from said Salt Reactor or from said Separator for adsorption of CO_{X} and/or NO_{X} in said Scrubber with said aqueous Scrubber aqueous phase. It is preferred to react said aqueous solution from said Scrubber with a metal salt solution in order to reduce the concentration of the metal(s) in said salt solution below their point of saturation in order to minimize fouling of said Scrubber with insoluble precipitate of said metal(s) CO₃ and/or NO_{2 or 3}. It is most preferred to add a dispersant to an aqueous recycle so as to minimize fouling of said Scrubber with insoluble precipitate of said metal(s) CO₃ and/or NO_{2 or 3}.
Transportation—In transportation, the ability to reduce a gaseous CO_{X} to a solid salt for either conversion to O₂ or disposal purposes has significant value to humanity. As presented previously:

CₙH₂ₙ₊₂+( 3/2n+½)O₂→nCO₂+(n+1)H₂O+Energy

More specifically, for gasoline (2,2,4 trimethyl pentane or n-Octane):

gasoline (Octane)+12½O₂→8CO₂+9H₂O+1,300 kcal

Therefore, an automobile obtaining 20 miles per gallon and a 15 gallon fuel tank produces about:
60 mph/20 mpg [symbol] (3 g)(5.8 lb./g)(454 gm/lb.)(/114)(M/gm Octane.)(8 M/M)(44 gm CO₂/M)≈24,400 gm CO₂/hr. 400 gm CO₂/mile 8,100 gm CO₂/gallon Octane, and for that automobile a 15 gallon fuel tank [symbol] 122,000 gm CO₂/tank, which is only near 3 times the original fuel weight of near 39,500 gm.
   A truck obtaining 4 mpg @ 60 mph and a 100 gallon fuel tank [symbol] 1,600 gm CO₂/mile and near 810,000 gm CO₂/tank of fuel, which is again about 3 times the original fuel weight of near 265,000 gm.

Converting CO₂ to CaCO₃ means for:
[symbol] An automobile at 20 mpg and a 15 gallon fuel tank storing near 277,000 gm of CaCO₃ ((122,000)( 100/44)) prior to refueling, which is about 6 times the original fuel weight, and
[symbol] A truck at 4 mpg and a 100 gallon fuel tank storing near 1,840,000 gm of CaCO₃ (810,000 gm)( 100/44) prior to refueling, which is again about 6 times the original fuel weight.

Converting CO₂ to MgCO₃ means for:
[symbol] An automobile at 20 mpg and a 15 gallon fuel tank storing near 240,000 gm of MgCO₃ ((122,000)( 85/44)) prior to refueling, and
[symbol] A truck at 4 mpg and a 100 gallon fuel tank storing near 1,565,000 gm of MgCO₃ (810,000 gm)( 85/44) prior to refueling.

Converting CO₂ to NaHCO₃ means for:
[symbol] An automobile at 20 mpg and a 15 gallon fuel tank storing near 190,000 gm of NaHCO₃ 022,000)( 68/44)) prior to refueling, and
[symbol] A truck at 4 mpg and a 100 gallon fuel tank storing near 1,252,000 gm of NaHCO₃ (810,000 gm)( 68/44) prior to refueling.

Converting CO₂ to KHCO₃ Means for:
[symbol] An automobile at 20 mpg and a 15 gallon fuel tank storing near 233,000 gm of KHCO₃ ((122,000)( 84/44)) prior to refueling, and
[symbol] A truck at 4 mpg and a 100 gallon fuel tank storing near 1,546,000 gm of NaHCO₃ (810,000 gm)( 84/44) prior to refueling.

It is preferred that the refueling station wherein a mode of transport obtains hydrocarbon, fossil, fuel have the capability of providing to said mode of transportation fresh water for said Scrubber. It is preferred that the refueling station wherein a mode of transport obtains hydrocarbon, fossil, fuel have the capability of taking from the mode of transport any stored aqueous phase from said Scrubber. It is preferred that the refueling station wherein the mode of transport obtains hydrocarbon, fossil, fuel have the capability of providing to said mode of transportation fresh metal salt. It is preferred that the refueling station wherein the mode of transport obtains hydrocarbon, fossil, fuel have the capability of taking from the mode of transport any unused metal salt and/or any metal-CO₃ and/or a metal-NO_{X} formed.

Catalysis—It is an embodiment to locate a metal catalyst in the exhaust of a hydrocarbon combustion engine or furnace prior to and/or after the Scrubber in order to minimize NO_{X} to the Scrubber and/or to the atmosphere. It is preferred that the metal(s) in said metal catalyst comprise at least one of platinum and rhodium
Metal Salt(s) Processing—It is an embodiment that the metals salt(s) comprise at least one selected from the group consisting of said: Scrubber, Salt Reactor, Separator, and any combination therein, be provided a means to an algae-type greenhouse or an ABR wherein the algae and/or plant growth therein is fed at least one of CO_{X} and/or NO_{2 or 3} as a food source. It is preferred that said solid phase from said Salt Reactor when located at the greenhouse be treated with an acid so as to release at least one of CO₂ and/or NO_{2 or 3} so as to provide the CO₂ and/or NO_{2 or 3} as a food source for the plant growth in the greenhouse. It is preferred that said acid be a sulfoxy acid. It is most preferred that said acid be sulfuric acid.

It is an embodiment that the solid phase from said Salt Reactor be used as a construction material. It is preferred that the solid phase from said Salt Reactor be used as a soil stabilizer. It is preferred that the solid phase from said Salt Reactor be used as a material in wallboard construction. It is preferred that the solid phase from said Salt Reactor be used as a material in marble manufacture.

It is preferred that the solid phase from said Salt Reactor be washed with water so as to reduce the concentration of NO_{2 or 3} in the solid phase.

It is most preferred that the solid phase from at least one selected from the group consisting of said: Scrubber, Salt Reactor, Separator, and any combination therein, be stored as a means of storing said CO_{X} and/or NO_{X} in a solid form.

It is most preferred that the solid phase from at least one selected from the group consisting of said: Scrubber, Salt Reactor, Separator, and any combination therein, be stored in the ocean or any body of water comprising an alkaline pH so as to maintain at least a portion of said CO_{X} and/or NO_{X} in a solid form.

Metal Salt(s) Processing—It is an embodiment that the metal salt(s) from the Scrubber be provided a means to an ABR wherein algal growth therein is performed with at least one of CO_{X} and/or NO_{2 or 3} as a food source. It is preferred that the metal salt(s) be reacted with an acid to release CO_{X} and/or NO_{X} prior to or within the ABR. It is preferred that the acid be sulfuric acid.
Aqueous Phase Processing—It is an embodiment that the aqueous phase from at least one selected from the group consisting of said: Scrubber, Salt Reactor, Separator, and any combination therein, be provided means of an algae-greenhouse or ABR wherein algae and/or plant growth therein is fed CO₂ and/or NO_{2 or 3} as a food source.

It is an embodiment that the aqueous phase from at least one selected from the group consisting of said: Scrubber, Salt Reactor, Separator, and any combination therein, be provided means of denitrification, as is known in the art, wherein facultative bacteria, reduce the NO_{2 or 3} in the aqueous phase to N₂. It is preferred that said means of denitrification comprise a carbon source for growth of said facultative bacteria. It is most preferred that the COD:N ratio within said denitrification means be between 6:1 and 3:1. It is an embodiment that the aqueous phase from said Salt Reactor be sent to an anaerobic biological means comprising (sulfur reducing bacteria) SRB bacteria, as are known in the art, wherein any sulfite, bi-sulfite, sulfate or bi-sulfate within the aqueous phase are reduced to sulfides by the SRB bacteria. In the operating scenario wherein anaerobic means are used to reduce any or either of said sulfite, bi-sulfite, sulfate or bi-sulfate, it is preferred that downstream of the SRB anaerobic means there be a facultative biological means comprising sulfur consuming bacteria, as are known in the art, to convert at least a portion of any H₂S, SO₂, and SO₃ to elemental sulfur. It is most preferred that said sulfur consuming bacteria comprise one of the species of the genus Thiobacillus, such as Thiobacillus denitrificans. It is most preferred that said sulfur consuming bacteria have a source of carbon.

It is most preferred that the aqueous phase from at least one selected from the group consisting of said: Scrubber, Salt Reactor, Separator, and any combination therein, be stored in the ocean or any body of water comprising an alkaline pH so as to maintain at least a portion of said CO_{X} and/or NO_{X} in a solid form.

It is preferred that the dissolved O₂ content within the aqueous phase of any facultative biological system be about 0.5 ppm O₂ or less. It is most preferred that the dissolved O₂ content within the aqueous phase of any facultative biological system be about 0.3 ppm O₂ or less.

It is most preferred that the carbon source for either denitrification or sulfide consuming bacteria be a form of waste water.

It is an embodiment to transport said precipitate and or said aqueous phase from at least one selected from the group consisting of said: Scrubber, Salt Reactor, Separator, and any combination therein, to at least one of: an algae greenhouse and a facultative biological reactor.

Algae Biological Reactor (ABR)—Algae assimilate soluble CO₂ and/or NO_{2 or 3} and not gaseous CO₂ and/or NO_{2 or 3}, ABR means is constrained by the water solubility and water solubility kinetics of CO₂ and/or NO_{2 or 3} water adsorption. As the absorption by algae of photons (light) is limited by photon aqueous phase penetration depth, which depends on the genus and specie of algae as well as algae concentration and photon availability, ABR means is constrained by algae specie, the depth of algae in water and photon availability. Most importantly, as algae only grow with the availability of photons, ABR means is constrained by light availability. As algae grow in relation to the Arrhenius Relationship, e.g. an about doubling of temperature corresponding to an about doubling of activity, temperature is a significant ABR operating parameter. As algae growth slows with increasing O₂ concentration in water, O₂ concentration is a parameter in ABR means. As algae require an operating pH range, pH is a parameter for ABR means. As algae require a source of Total Organic Carbon (TOC), soluble TOC is a parameter for ABR means. As algae require nutrients, the concentration of nutrients is a parameter for ABR means. As algae production of H₂ is significantly affected by the concentration of O₂ and of S in water, the concentration of O₂ and of S are significant parameters in ABR means to produce H₂. It is preferred for the production of H₂ that an ABR comprise an about absence of O₂, wherein at least one of S and N₂ are in an about absence in the algal environment, such that at least one of the absence(s) promote the algae in the ABR to produce H₂. And, as algae production is enhanced with immobilization, means of immobilization or surface adherence for colonization is a parameter for ABR means.

It is an embodiment that the ABR comprise algae. It is preferred that the algae in the greenhouse or the ABR be at least one algae selected from the group consisting of: Anabaena cylindrical, Bostrychia scorpioides, Botycoccus braunii, Chaetoceros muelleri, Chlamydomonas moeweesi, Chlamydomonas reinhardtii, Chlorella pyrenoidosa, Chlorella vulgaris, Chlorella vulgaris Beij, Dunaliella bioculata, Dunaliella sauna, Dunaliella tertiolecta, Euglena gracilis, Isochysis galbana, Isochysis galbanais micro, Nannochloris sp., Nannochloropsis sauna, Nannochloropsis sauna Nannochloris oculata—N. oculata, N. atomus Butcher, N. maculata Butcher, N. gaditaa Lubian, N. oculata, Neochloris oleoabundans, Nitschia communis, Parietochloris incise, Phaeodactylum tricornutum, Pleurochrysis carterae, haptophyta, prymnesiophyceae, Porphyridium cruentum, Prymnesium parvum, Scenedesmus dimorphus, Scenedesmus obliquus, Scenedesmus quadricauda, Schenedesmus dimorphus, Spirogyra sp., Spirulina maxima, Spirulina platensis, Spirulina sp., Synechoccus Tetraselmis chui, Tetraselmis chui, Tetraselmis maculate, Tetraselmis suecica, Botycoccus braunii, and any combination therein. It is most preferred that the algae in the greenhouse or the ABR be at least one algae selected from the group consisting of Botryococcus braunii strains, Chlamydomonas reinhardtii, Chlorella vulgaris, Anabaena cylindrical, Chlamydomonas rheinhardii, Chlorella pyrenoidosa, Chlorella vulgaris, Dunaliella bioculata, Dunaliella sauna, Euglena gracilis, Porphyridium cruentum, Prymnesium parvum, Scenedesmus dimorphus, Scenedesmus obliquus, Scenedesmus quadricauda, Spirogyra sp., Spirulina maxima, Spirulina platensis, Synechoccus sp., Tetraselmis maculate, and any combination therein.

It is preferred that the algae is at least one of non-pathogenic, non-opportunistic, low-virulence factor, and any combination therein. It is an embodiment that the algae be mutant.

It is preferred that the algae in the ABR be selectively cultured to convert at least one selected from the group consisting of CO₂ and H₂O into O₂ and a hydrocarbon, CO₂ and H₂O into a protein, CO₂ and H₂O into H₂, and any combination therein. It is an embodiment the algae in the ABR be mutant.

It is an embodiment that the ABR have a photon penetration depth within the aqueous phase to the algae of 100 cm or less. It is preferred that the ABR have a photon penetration depth within the aqueous phase to the algae of 10 cm or less. It is a most preferred embodiment that the ABR have a photon penetration depth within the aqueous phase to the algae of 5 cm or less. It is most preferred that the algae in the ABR have a reduced chlorophyll content so as to improve photon (light) penetration in the ABR. It is preferred that the photon concentration in the ABR is greater than 10 W/m² and equal to or less than the photon saturation point for at least one specie of algae in the ABR. It is an embodiment that the photoperiod comprise a time of light and dark which is 20 hours of light and 4 hour of dark to 4 hours of light and 20 hours of dark. It is preferred that the photoperiod comprise 12 hours of light and 12 hours of dark.

It is preferred that at least a portion of the Gas flow is in aqueous solution in the ABR. It is most preferred that at the Gas flow is supplied to the aqueous solution in the ABR from a Scrubber. It is preferred that Gas flow is supplied to the ABR as a gas. It is preferred that the Gas flow be supplied to the ABR as a mixture with air. It is preferred that the Gas flow be introduced into the ABR via means to reduce or minimize bubble size. It is most preferred that the Gas flow be introduced into the ABR via a membrane type of material, as is known in the art. It is preferred that the Gas flow be dispersed in the ABR via a tube made of a membrane type material, as is known in the art of gas transfer. It is preferred that the Gas flow be dispersed in an ABR via a tube comprising holes (gas tube). It is preferred that the Gas flow be dispersed in an ABR via a gas tube, wherein the gas tube comprises a membrane type material, such that the Gas flow is forced through the membrane material into the aqueous phase. It is preferred that the Gas flow be dispersed in an ABR via a tube made of membrane type material or a gas tube surrounded by membrane type material and that the Gas flow and tube sizing be such that Gas flow pressure within the tube can be managed. It is most preferred that the Gas flow pressure within the tube be about the same from end to end. It is most preferred that the membrane of the gas tube be such that gas flow into the aqueous solution is about the same from end to end and regardless of water depth and/or pressure. It is most preferred that the membrane of the gas tube be such that the holes for gas flow into the aqueous solution are sized so as to about compensate for hydrostatic pressure within the aqueous phase such that gas flow for is about the same from end to end and regardless of water depth and/or pressure. It is most preferred that the tube be coaxial to and within an ABR, wherein the ABR comprises a tubular shape. The concentration of CO₂ in the Gas flow introduced to the ABR is preferred in the range of 0.04 to 100 percent.

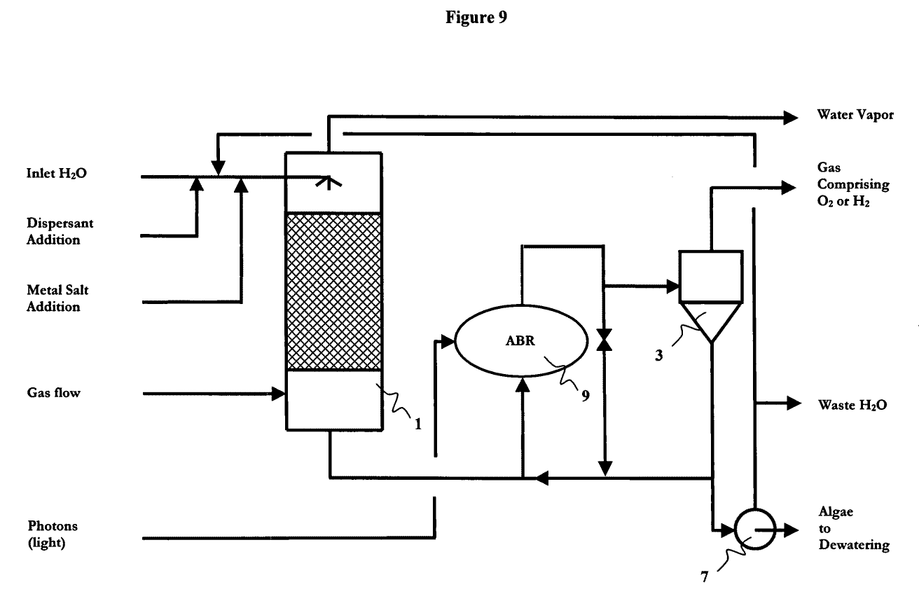

It is preferred that the Gas flow introduced into the ABR be introduced into the ABR in a pattern so as to minimize shearing of the algae within the ABR while providing mixing of ABR contents. It is preferred that the Gas flow introduced into the ABR be introduced into a tubular shaped ABR in a manner consistent with the size of the ABR to create mixing of the aqueous solution within the ABR. It is most preferred that the mixing transfer algae to and from the side of the ABR nearest the source of light to the ABR. It is preferred that the Gas flow introduced into the ABR be introduced into the ABR in a manner consistent with the size of the ABR to create turbulent flow of the aqueous solution within the ABR. It is most preferred that the Gas flow introduced into a tubular ABR be introduced in a location within the ABR such that the means of Gas flow introduction minimally inhibits photon transfer in the aqueous phase. In the case of a tubular ABR, it is preferred that a tubular membrane be used to introduce the Gas flow and that the tubular membrane be located on the wall of the tubular ABR. In the case of a tubular ABR wherein the photon tube is in the center of the ABR, it is most preferred that the gas tube encircle the photon tube on the wall of the tubular ABR from a beginning point located on one side of the center of the length of the tubular ABR to another point on the other side of the center of the length of the tubular ABR. It is most preferred that said beginning point be near one end of the tubular ABR. It is most preferred that said another point be near the opposite end of the tubular ABR from beginning point. In the case of a Continuous Stirred Tank Reactor (CSTR) ABR, Gas flow may enter the CSTR at any location, be that in or near the base, from or near the walls, via tubes or media in the aqueous solution as depicted in FIG. 9, and any combination therein.

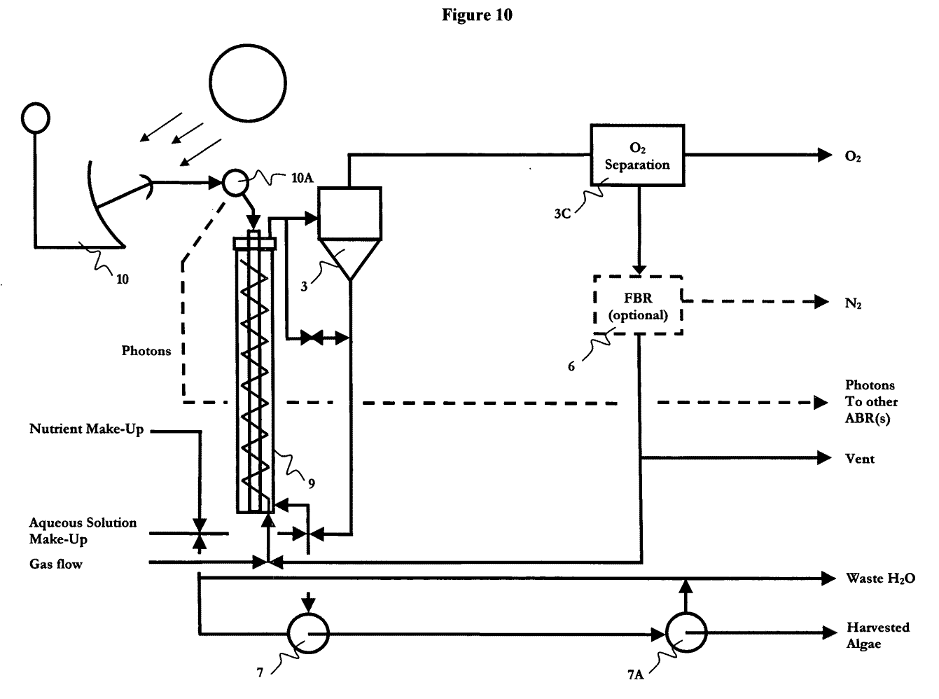

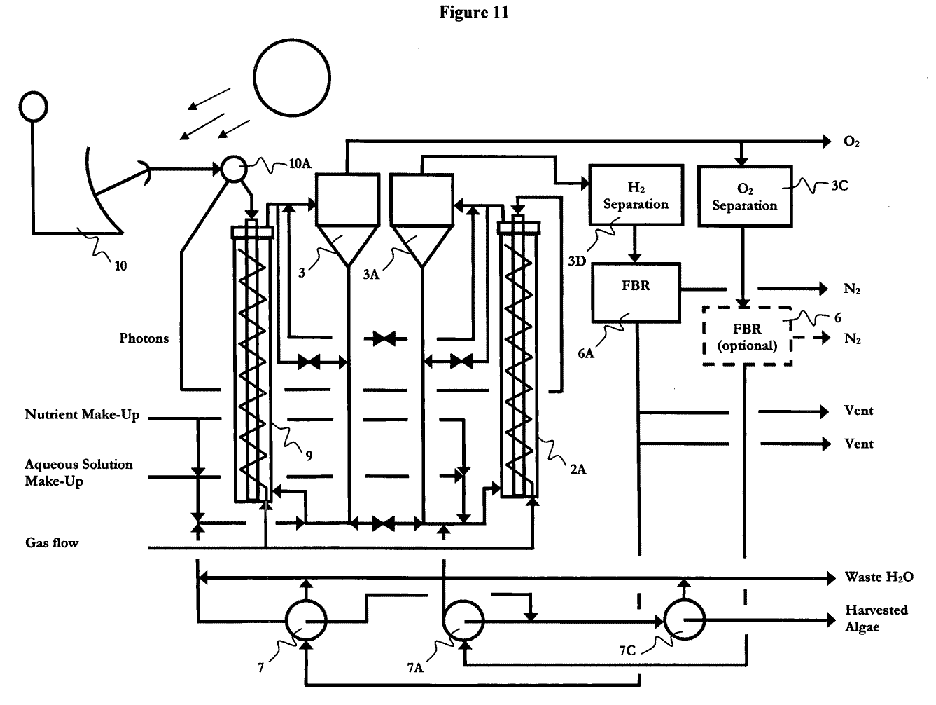

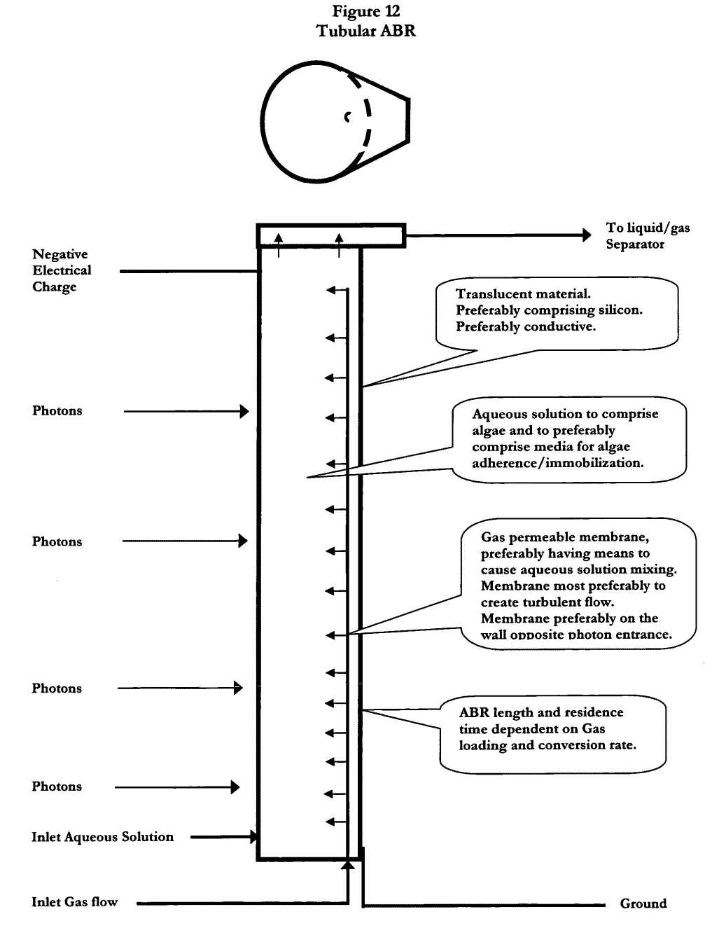

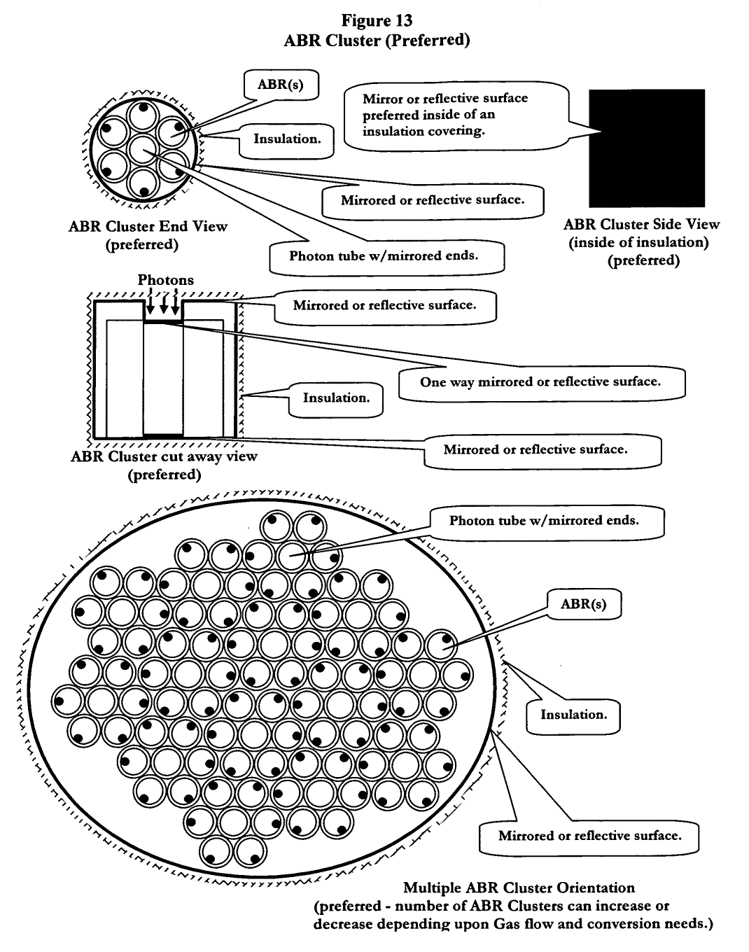

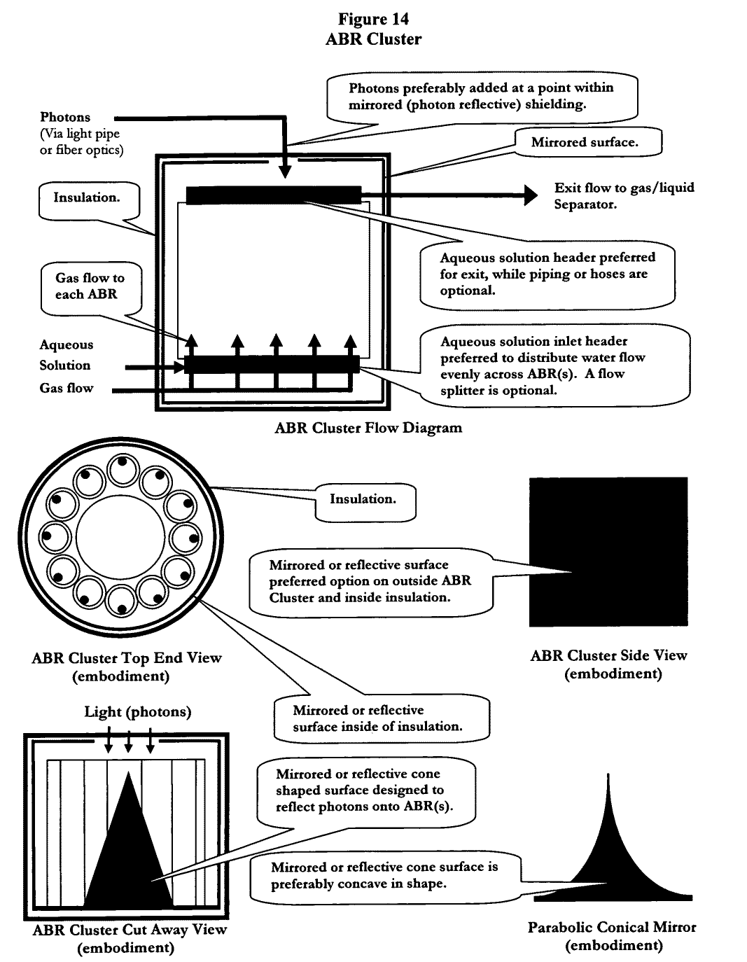

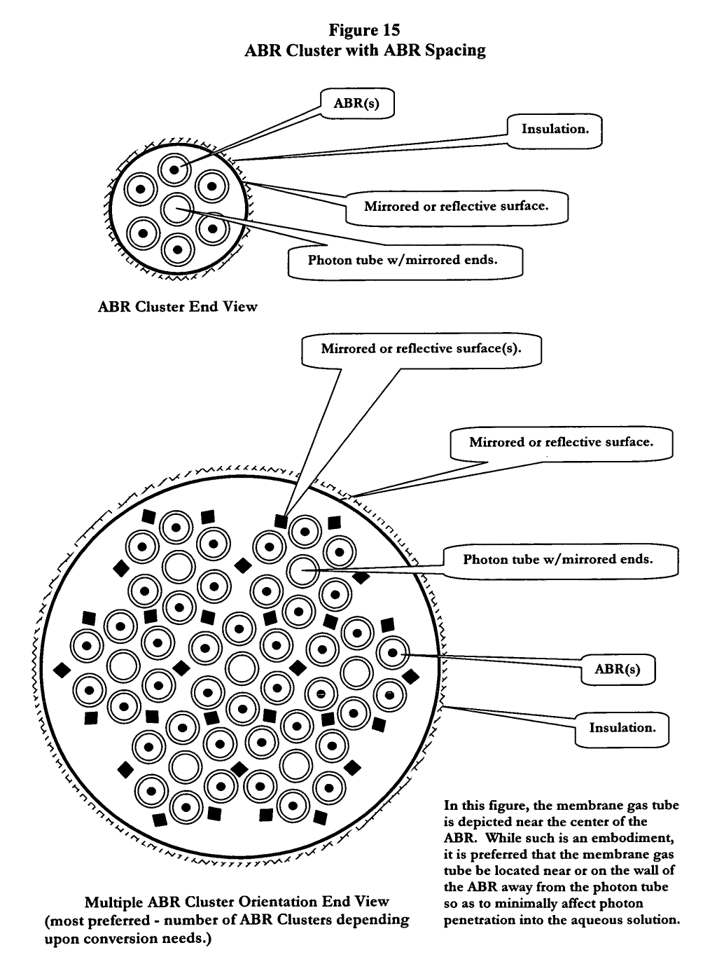

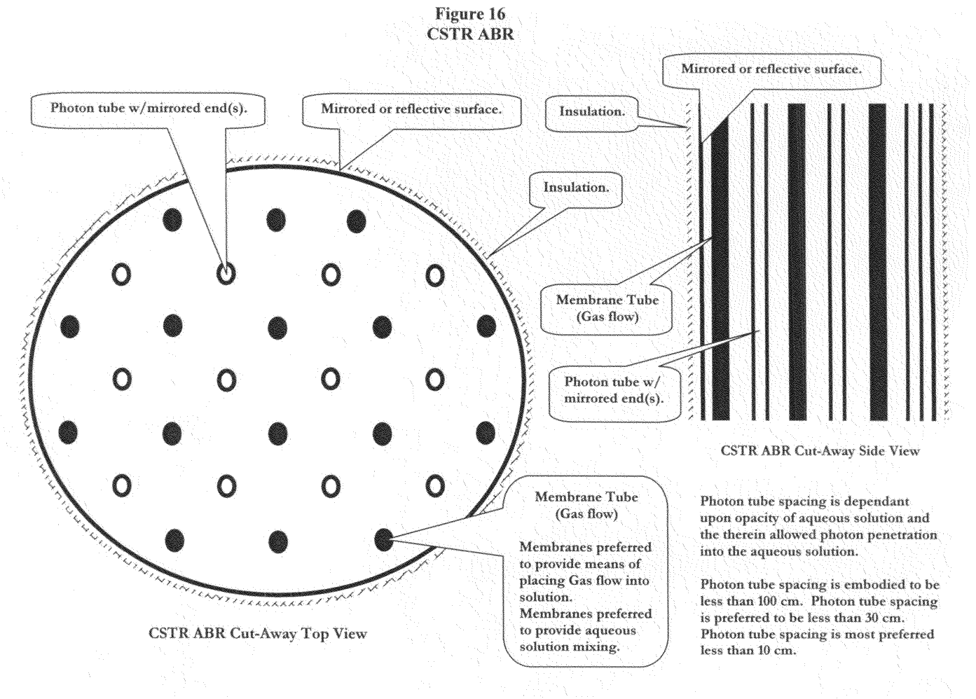

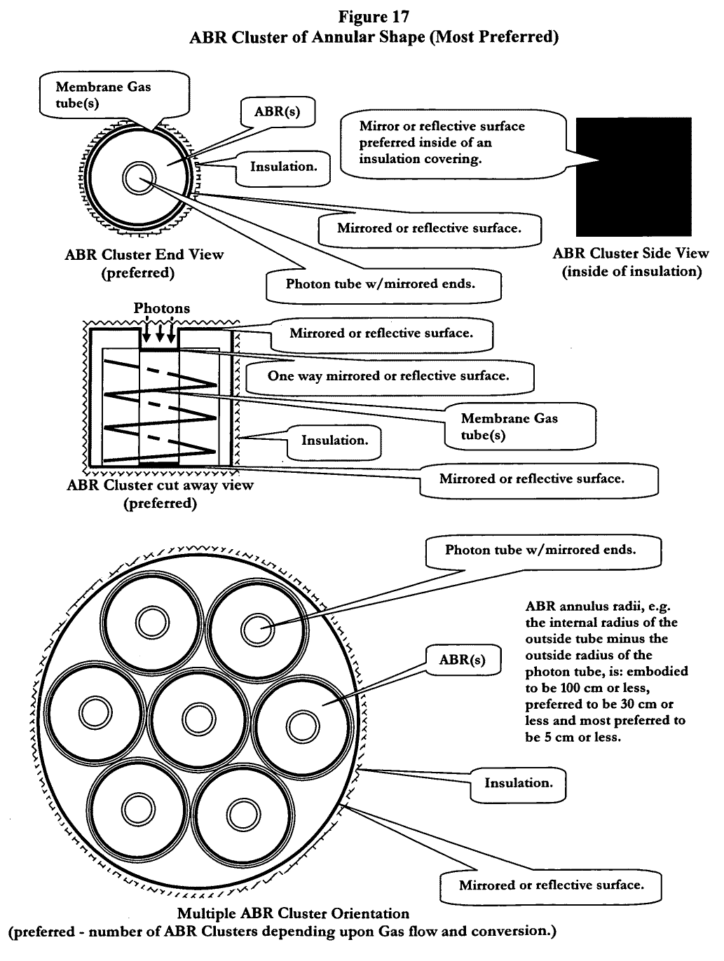

It is preferred that the ABR be made of tubular construction. It is preferred that there be a number of tubular ABR(s). It is preferred that the ABR(s) be of tubular shape and comprise a diameter of 5 cm or less. It is preferred that the ABR(s) comprises at least one of: silicon, glass, carbonate, a conductive material, metal, and any combination therein. It is most preferred that the tubular ABR be of annular construction such that the ABR is a tube within a tube, wherein the photons enter the ABR via the center tube and the ABR aqueous solution comprise the annulus or radii between the outer tube and the inner tube as depicted in FIG. 10.

It is most preferred that the ABR be of CSTR Design. It is most preferred that the CSTR ABR comprise a number of photon tubes. It is most preferred that photon tube spacing in the CSTR ABR be such that light (photons) may penetrate to the algae. It is most preferred that the Gas flow introduction to a CSTR ABR be such that mixing of the aqueous phase is maintained. It is preferred that the Gas flow introduction to a CSTR ABR be such that mixing of the aqueous phase is maintained such that the concentration of CO_{X} at any vertical level in the CSTR ABR not vary by more than 50 percent. It is most preferred that the Gas flow introduction to a CSTR ABR be such that mixing of the aqueous phase is maintained such that the concentration of CO_{X} at any vertical level in the CSTR ABR not vary by more than 25 percent. It is an embodiment that the photon tube(s) in a CSTR ABR be no more than 100 cm apart. It is preferred that the photon tube(s) in a CSTR ABR be no more than 30 cm apart. It is most preferred that the photon tube(s) in a CSTR ABR be no more than 10 cm apart.

It is preferred that the ABR(s) be made of a translucent material. It is preferred that the ABR(s) material of construction comprise Silicon. It is preferred that the ABR(s) material of construction comprise glass. It is preferred that the ABR(s) material of construction comprise carbonate. It is preferred that the ABR(s) material of construction comprise a metal so that an electric charge may be placed upon the wall of the ABR(s). It is most preferred that an electric charge be placed upon the wall surface of the ABR(s) thereby creating a zeta potential on the wall surface of the ABR(s) to reduce algal tackification to the wall surface of the ABR(s). It is preferred that the ABR(s) have a means of vibration. It is preferred that the ABR(s) have a means of vibration to reduce algal tackification to the wall surface of the ABR(s). It is preferred that the ABR(s) comprise a means of ultrasonics as a means to reduce algal tackification to the wall surface of the ABR(s), as well as reduce algae agglomeration. In the means of ultrasonics, it is most preferred that at least one of the ultrasound amplitude and frequency be limited so that the energy of ultrasonics does not affect algae cell viability.

It is an embodiment that light be made available to the ABR(s). It is preferred that light be transferred via at least one mirror to the ABR(s). It is most preferred that light be concentrated and transferred via at least one mirror to at least one ABR(s).

It is preferred that at least one photon (light) collector concentrate light as is known in the art. It is preferred that the light collector(s) have an ability to track the Sun or change position so as to maintain an optimum position of photon collection in relation to the position of the sun, as is known in the art of light collection. It is preferred that the light collector comprises at least one reflective or mirrored surface. It is preferred that the light collector be of dish type design concentrating light to the focal point of the dish, as is known in the art of light collection. It is preferred that the light from a number of light collectors be transferred to a distribution point, wherein from the spherical shaped distribution point light is transferred to at least one ABR. It is preferred that the distribution point comprise a spherical shape. It is preferred that the distribution point comprise a mirrored surface. It is preferred that the means of transfer be of tube shape, wherein the inside surface of the tube comprises a reflective or mirrored surface so as to reflect light (photons). It is preferred that the mirrored tube(s) transfer photons down the inside of the tube to at least one ABR. It is preferred that said tube comprise a pressure of less than 1 atmosphere. It is most preferred that the light be placed in a fiber optic cable, as is known in the art, for transfer of the light to at least one ABR. It is preferred that the fiber optic cable comprises a reflective or mirrored surface so as to reflect light. It is preferred that an ultraviolet light filter reduce at least a portion of the ultraviolet light from the concentrated light prior to transfer to at least one ABR. It is preferred that the concentrated light be separated so as to emit into at least one ABR.

It is preferred that at least a portion of the hydrocarbon product of the algae or at least a portion of the algae itself from within at least on ABR be used to generate electrical energy. It is preferred that at least a portion of the hydrocarbon product of the algae or at least a portion of the algae itself from within at least on ABR be used to generate electrical energy and that at least a portion of the electrical energy be used to produce photons for at least one of the ABR.

It is preferred that light (photons) be emitted upon and into at least one ABR. It is preferred that photons be placed upon a number of ABR. It is preferred that light be placed upon a number of tubular ABR such that the tubular ABR are arranged around the placement of light (this is termed herein as an ABR Cluster). It is preferred that an ABR Cluster be arranged such that the ABR(s) in the ABR Cluster are side-by-side and not end-to-end so as to form around the placement of light. It is preferred that the placement of light be within a cylinder or tube (herein after a cylinder or tube transferring photons among and to the ABR(s) is termed a photon tube).

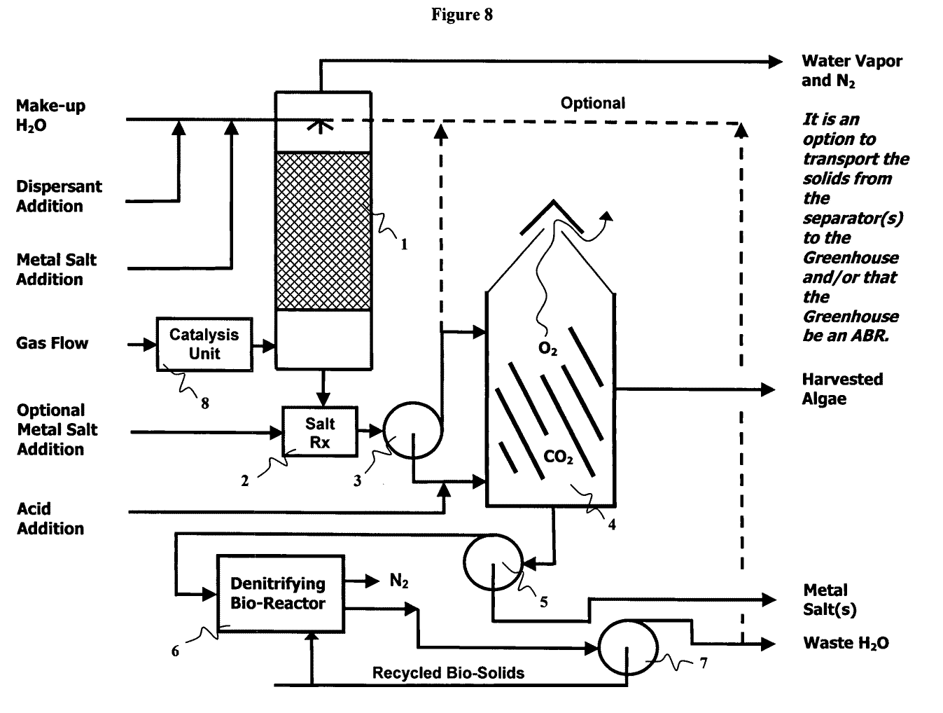

It is preferred that the ABR Cluster comprises the photon tube in the center, wherein photons are distributed to the ABR(s). It is preferred that a number of ABR and photon tube be arranged such that there is two ABR between each of two photon tubes, as depicted in FIG. 8. It is preferred that the photon tube comprises a translucent material and comprises at least one of: a one way mirror at one end, the one way mirror allowing photon entrance into the photon tube while reflecting photons from leaving the same end, and a reflective or mirrored surface at the end opposite the end of photon entrance. It is an embodiment that the ABR Cluster comprises space between the ABR(s), wherein the space between the ABR(s) allows photons from the photon tube to pass between the ABR(s), such that the photons which pass between the ABR(s) are reflected from a reflective or mirrored surface onto the side of the ABR(s) which does not face the photon tube. It is preferred that the ABR Cluster comprises at least one of a one way mirror at one end, the one way mirror allowing photon entrance into the ABR Cluster while reflecting photons from leaving the same end, a reflective or mirrored surface at the end opposite the end of photon entrance, and a conical shaped reflective or mirrored surface at the end opposite the end of photon entrance.

It is most preferred that the photon tube comprise a fiber optic cable.

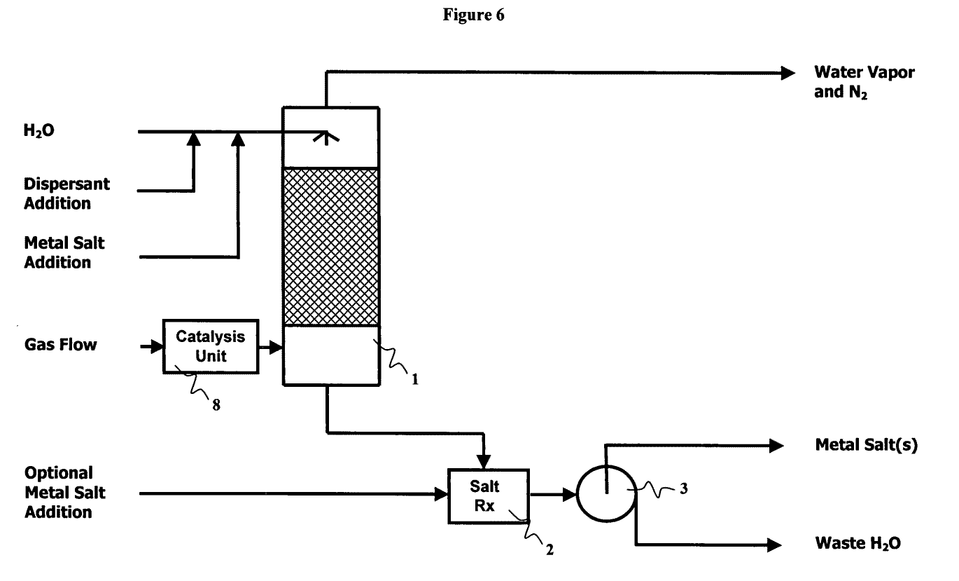

It is preferred that the number of ABR in an ABR Cluster be between 4 and 12. It is most preferred that the number of ABR in an ABR Cluster be 6. It is most preferred that the diameter of the tubular ABR and the diameter of the photon tube be about the same. It is preferred that there be a number of ABR Cluster. It is most preferred that the number of ABR Cluster be placed side-to-side so as to form a hexagonal honeycomb shape when viewed from the end, as depicted in FIG. 6, 7 or 8.

It is an embodiment that photons be placed between the ABR tubes forming the ABR Cluster, wherein the photons are released into one end of the ABR Cluster between the ABR(s). It is an embodiment that the photons placed between the ABR tubes forming the ABR Cluster at one end of the ABR Cluster, wherein a reflective or mirrored surface is located at the opposite end of the ABR Cluster. It is preferred that the reflective or mirrored surface be conical in shape.

It is most preferred that each ABR Cluster or a number of ABR Cluster be at least partially enclosed in a reflective or mirrored means to reflect (photons) light from or near the ABR(s) into the ABR(s).

It is preferred that a number of ABR Cluster be located in a unit or apparatus.

It is preferred that a number of CSTR ABR be located in a unit or apparatus.

It is preferred that each ABR comprise means of removal from a unit comprising at least one ABR, wherein the at least one ABR comprise a means of sealing the inflow or outflow of at least one of the aqueous solution and the Gas flow, as needed. It is preferred that each ABR(s) within an ABR Cluster comprise a means of removal and replacement. It is most preferred that the ABR(s) comprise a sealing of at least one of the inflow gas and inflow aqueous solution, and a sealing of the outflow aqueous solution, such that the ABR is easily removed and replaced.

It is preferred that there be placed within at least one ABR a means of measuring light intensity, as is known in the art of light measurement. It is most preferred that the amount of light within an ABR be between 10 W/m² irradiance and photosaturation for an algae within the ABR. It is preferred that a control loop be placed within the light transfer means so as to obtain an input signal from the light intensity measuring means and reduce or filter light to the ABR when light intensity is near photosaturation for an algae within the ABR.

It is an embodiment that the temperature within the ABR(s) is between 17 and 70° C. It is preferred that the temperature within the ABR(s) is within a 5° C. range of temperature, wherein the 5° C. range of temperature is between 17 and 70° C. It is preferred that the ABR(s) be insulated from ambient temperature with the materials of insulation as is known in the art of insulation. It is most preferred that each ABR Cluster or number of ABR Cluster in a unit be insulated from the ambient temperature with materials of insulation as is known in the art of insulation. It is preferred that a temperature sensor be located within at least one ABR or ABR Cluster to measure the water temperature either just before each ABR, within each ABR or after each ABR. It is preferred that at least one of a water cooling or a water heating device, as is known in the art of water heating and cooling, be placed so as to perform at least one of heating and cooling of the water entering at least one ABR or ABR Cluster.

It is preferred that the O₂ aqueous solution concentration in each ABR or ABR Cluster is less than 40 percent. It is preferred to reduce the concentration in the Gas entering each ABR or ABR Cluster by diluting the Gas with air. It is an embodiment to vent the ABR or ABR Cluster in order to control the ABR O₂ aqueous solution concentration.

As CO₂ creates carbonic acid in aqueous solution, it is preferred to have a means of pH control for at least one ABR or ABR Cluster. It is preferred that the pH in the ABR be between 6 and 10. It is most preferred that the pH in the ABR be between 8 and 9. It is preferred that the aqueous solution comprise at least one of a base and a buffer. It is preferred that the aqueous solution comprises at least one selected from the group consisting of: hydroxide, bi-carbonate, magnesium, and any combination therein. It is preferred that there be a pH meter to measure pH within at least one ABR or ABR Cluster. It is preferred to have a pH control loop wherein a base is added to the aqueous solution for at least one ABR or ABR Cluster.

As algae need nutrients to grow, it is preferred that within the ABR aqueous solution is a nutrient concentration. It is preferred that the aqueous solution comprise at least one selected from the group consisting of: a phosphate, ammonium hydroxide, sulfur, iron, a carbon compound, and any combination therein. It is most preferred that a unit adds to the aqueous solution for at least one ABR or at least one ABR Cluster at least one nutrient selected from the group consisting of: phosphate, ammonia, nitrogen oxide, iron, sulfur, a carbon compound and any combination therein.

It is preferred to operate an ABR or an ABR Cluster with a reduced concentration of O₂ along with a reduced concentration of S and/or of N₂ in ABR aqueous solution in order for the algae in the aqueous solution to produce H₂ instead of O₂. It is preferred to operate an ABR or an ABR Cluster wherein the concentration of O₂ is reduced and at least one of S and N₂ is reduced enough to facilitate in each ABR or ABR Cluster the production of H₂ instead of O₂. It is an embodiment to operate at least one ABR or ABR Cluster in the production of O₂ and at least one ABR or ABR Cluster in the production of H₂.

As algae growth is best performed with immobilization or agglomeration of the algae, it is an embodiment that the algae within at least one ABR have the ability to adhere to a media within the ABR aqueous solution. It is an embodiment that the media be hydrophobic. It is an embodiment that the media have a density of between 0.7 and 1.3. It is preferred that the media have a density of about 1.0. It is a most preferred an embodiment that the material of the media comprise a material which is resistant to acids. It is a most preferred an embodiment that the material of the media comprise a material which is resistant to bases. It is an embodiment that the materials of the media comprise a polymer as is known in the art of polymer science. It is an embodiment that the media have a rough surface for algal adherence.

Combustion of H₂ and O₂—It is a most preferred embodiment to utilize at least a portion of at least one of the H₂ produced in the ABR(s) and the O₂ produced in the ABR(s) as an energy source for the operation of at least one ABR or at least one ABR Cluster. It is a most preferred embodiment to utilize at least a portion of at least one of the H₂ produced in the ABR(s) and the O₂ produced in the ABR(s) in combustion as an energy source to heat the water entering at least one ABR or at least one ABR Cluster. It is a most preferred embodiment to utilize at least a portion of at least one of the H₂ produced in the ABR(s) and the O₂ produced in the ABR(s) as an energy source to drive a generator to power the separation of at least one of O₂ from a gas and H₂ from a gas. It is a most preferred embodiment to utilize at least a portion of at least one of the H₂ produced in the ABR(s) O₂ produced in the ABR(s) in combustion as an energy source to drive a generator to power the operation of at least one ABR or at least one ABR Cluster. It is preferred that at least a portion of said H₂ and/or at least a portion of said O₂ is combusted to create photons of said algae and/or at least one of said ABR.
Denitrifying Bacteria—It is an embodiment that the aqueous phase from the Scrubber or from the ABR be provided means of denitrification, as is known in the art, wherein facultative bacteria, as are known in the art, reduce the NO_{2 or 3} in the aqueous phase to N₂. It is preferred to perform denitrification in a Facultative Biological Reactor (FBR). It is preferred that the means of denitrification comprise a carbon source for growth of the facultative bacteria. It is most preferred that the COD:N ratio within the denitrification means be between 6:1 and 3:1. It is an embodiment that the aqueous phase be sent to an anaerobic biological means comprising sulfite reducing bacteria (SRB), as are known in the art, wherein any sulfite, bi-sulfite, sulfate or bi-sulfate within the aqueous phase are reduced to sulfides by the SRB. In the operating scenario wherein anaerobic means are used to reduce any or either of the sulfite, bi-sulfite, sulfate or bi-sulfate, it is preferred that downstream of the SRB anaerobic means there be a facultative biological means comprising sulfur consuming bacteria, to convert at least a portion of any H₂S, SO₂, and SO₃ to elemental sulfur.

It is a preferred embodiment that the aqueous phase be reacted with sulfur consuming bacteria wherein any sulfite, bi-sulfite, sulfate or bi-sulfate within the aqueous phase are reduced to sulfides by the SRB.

It is most preferred that the sulfur consuming bacteria comprise Thiobacillus, such as Thiobacillus denitrificans.

It is most preferred that the sulfur consuming bacteria have a source of carbon.

It is preferred that the denitrifying bacteria be at least one of non-pathogenic, non-opportunistic, low-virulence factor, and any combination therein.

It is preferred that the dissolved O₂ content within the aqueous phase of any facultative biological system be about 0.5 ppm O₂ or less. It is most preferred that the dissolved O₂ content within the aqueous phase of any facultative biological system be about 0.3 ppm O₂ or less.

It is most preferred that the carbon source for either denitrification or sulfide consuming bacteria be a form of waste water.

It is an embodiment that the aqueous phase of the FBR perform facultative denitrification of NO₂ and NO₃. It is most preferred that the denitrification comprise at least one of: the genera selected from the group consisting of: Pseudomonas, Bacillus, and Achromobacter, and any combination therein. It is most preferred that the denitrification be performed with at least one selected from the group consisting of Thiobacillus, such as Thiobacillus denitrificans.

Sulfur Consuming Bacteria—It is an embodiment that the liquid exiting the ABR be reacted in an FBR, wherein the FBR comprises bacteria which metabolize or consume sulfides and/or sulfur oxides into their biomass. It is a preferred embodiment that the aqueous solution or the liquid comprise at least one selected from the group consisting of: gram-negative bacteria from the beta or gamma subgroup of Proteobacteria, obligate autotrophs, Thioalkalovibrio, strain Al-2, Thioalkalobacter, alkaliphilic heterotrophic bacteria, Pseudomonas strain ChG 3, Rhodococcus erythropolis, Rhodococcus rhodochrous, Rhodococcus v., Nocardia erythropolis, Nocardia corrolina, other Nocardia sp., Pseudomonas putida, Pseudomonas oleovorans, Pseudomonas species, Arthrobacter globiformis, Arthobacter Nocardia paraffinae, Arthrobacter paraffineus, Arthrobacter citreus, Arthrobacter luteus, other Arthrobacter sp., Mycobacterium vaccae JOB, Mycobacterium Acinetobacter sp., Acinetobacter sp., Corynebacterium sp., Corynebacterium sp., Thiobacillus ferrooxidans, Thiobacillus intermedia, Thiobacillus sp., Shewanella sp., Micrococcus cinneabareus, micrococcus sp., Bacillus sulfavortare, bacillus sp., Fungi, White wood rot fungi, Phanerochaete chysosporium Phanerochaete sordida, Trametes trogii, Tyromyces palustris, white wood rot fungal sp., Streptomyces fradiae, Streptomyces globivorus, Streptomyces sp., Saccharomyces cerrevisiae, Candida sp., Cryptococcus albidus, yeasts and algae. It is most preferred that the aqueous phase of the FBR comprise at least one species of the genus Thiobacillus and the species therein of Thiobacillus denitrificans.

It is most preferred that the sulfur consuming bacteria is at least one of non-pathogenic, non-opportunistic, low-virulence factor, and any combination therein.

Separation—It is an embodiment to perform gas/liquid and liquid/solids separation means.

It is preferred to perform gas/liquid separation means, wherein the effluent aqueous solution from the ABR(s) is at least partially separated into a gas and a liquid. It is most preferred that the gas/liquid separation means comprises cyclone separation. It is preferred that at least a portion of the separated liquid is returned to the aqueous solution in the ABR(s). It is preferred that at least a portion of the separated liquid be further processed for bacterial wasting or for algae harvesting. In order to facilitate gas concentrations in the aqueous solution, it is preferred that there be a gas/liquid separation by-pass for ABR(s) aqueous solution effluent, wherein the aqueous solution effluent is returned to the aqueous solution in the ABR(s).

It is an embodiment to separate the O₂ from the ABR vent or the separated gas. It is preferred to perform the O₂ separation with at least one selected from the group of membrane separation, vacuum and/or pressure swing adsorption, cryogenic distillation, and any combination therein. In the case wherein the ABR(s) is producing H₂, it is preferred to separate the H₂ from the ABR vent or the separated gas. It is preferred to perform the H₂ separation with at least one selected from the group of: membrane separation, vacuum and/or pressure swing adsorption, cryogenic distillation, and any combination therein. It is a most preferred embodiment to utilize at least a portion of at least one of the H₂ and the O₂ from the ABR Cluster(s) in the combustion of H₂ with O₂ as the oxidizer, wherein the combustion comprises an energy source for the operation of at least one ABR or at least one ABR Cluster. It is a most preferred embodiment to utilize at least a portion of at least one of the H₂ and the O₂ as an energy source to heat the water entering at least one ABR or at least one ABR Cluster. It is a most preferred embodiment to utilize at least a portion of at least one of the H₂ and the O₂ as an energy source to drive a generator to power the O₂ separation. It is a most preferred embodiment to utilize at least a portion of at least one of the H₂ and the O₂ as an energy source to drive a generator to power the operation of at least one ABR or at least one ABR Cluster.

It is preferred that liquid/solids separation means be as is known in the art of water treatment. It is preferred that the liquid/solids separation means comprise one of clarification, thickening, filtration, centrifugation.

It is preferred to perform liquid/solids separation of effluent from an FBR. It is preferred to separate the FBR effluent into mostly FBR aqueous phase and mostly FBR biomass. It is preferred to further separate the FBR biomass into bacteria solids and sulfur. It is preferred that the further separation be performed via centrifugation.

It is preferred to separate the aqueous solution or the liquid into mostly an aqueous phase and mostly a solids phase, wherein the solids phase comprises algae. It is preferred that the aqueous phase be transferred to the aqueous solution in the ABR(s).

It is preferred to perform algae separation from the liquid by means of liquid/solids separation, e.g. gravity (clarification or thickening), filtering or centrifugation, as is known in the art of water treatment. It is most preferred to reduce the amount of liquid with the algae by means of centrifugation, a belt filter press or a drying bed, as is known in the art.

It is most preferred to condition at least one of the bacteria and the algae for liquid/solids separation and/or reducing the liquid concentration in a solids with at least one selected from the group consisting of a: cationic coagulant, quaternized cationic coagulant, cationic polyacrylamide, quaternized polyacrylamide, poly(DADMAC), poly(DADMAC) comprising a molecular weight of at least 1,000,000, poly(epi-DMA), poly(epi-DMA) comprising a molecular weight of at least 500,000, chitosan cationic polymer, quaternized chitosan polymer, starch cationic polymer, quaternized starch polymer, and any combination therein.

It is preferred in the case wherein algae is grown in the ABR(s) on a media, to first treat the algae on media with an acid to remove the algae from the media prior to separation of the algae from the liquid. It is preferred that the acid be carbonic or sulfuric.

Algae Harvesting—It is preferred to harvest the algae grown in the ABR(s). It is preferred to harvest the algae by liquid/solids separation means. It is preferred that the harvested algae be used as a protein in food applications or in animal feed. It is preferred that the harvested algae be further processed to obtain hydrocarbon oil(s) from the harvested algae. It is preferred that the harvested algae be used as a fertilizer. It is preferred that the harvested algae be used as a combustion fuel. It is preferred that the algae is used as at least one selected from the group consisting of a: protein in food applications, animal feed, hydrocarbon oil(s), combustion, fertilizer, and any combination therein.
Apparatus for Manufacturing Plants and Process Flow Paths—It is a preferred embodiment that an apparatus comprise at least one source of Gas Flow and at least one Scrubber having a source of water flow form a manufacturing plant and/or process flow path, wherein said source(s) of Gas Flow is upstream of said Scrubber(s) and wherein the water in said Scrubber(s) comprises at least one of: a dispersant and a dispersant in combination with a metal salt. It is preferred that said metal salt comprise a Group IA or IIA metal salt. It is most preferred that at least one unit add said dispersant and/or said metal salt to said water in said Scrubber(s) and/or to the water prior to entering said Scrubber(s).

It is a preferred embodiment that an apparatus comprise at least one source of Gas Flow, at least one Scrubber having a source of water flow and at least one Separator form a manufacturing plant and/or process flow path, wherein said source(s) of Gas Flow is upstream of said Scrubber(s) and said Scrubber(s) is upstream of said Separator(s), wherein the water in said Scrubber(s) comprises at least one of: a dispersant and a dispersant in combination with a metal salt, and wherein the solid phase from said Separator(s) comprises a metal salt comprising at least one of CO₃, NO₂ and NO₃. It is preferred that said metal salt comprise a Group IA or IIA metal salt. It is most preferred that at least a portion of the aqueous phase from said Separator(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one source of Gas Flow, at least one Scrubber having a source of water flow, at least one Salt Reactor and at least one Separator form a manufacturing plant and/or process flow path, wherein said source(s) of Gas Flow is upstream of said Scrubber(s), said Scrubber(s) is upstream of said Salt Reactor(s) and/or said Separator(s), wherein the water in said Scrubber(s) comprises at least one of: a dispersant and a dispersant in combination with a metal salt, wherein said Salt Reactor(s) forms from the reaction of an aqueous solution with metal salt a metal-CO₃ salt, and wherein the solid phase from said Separator(s) comprises a metal salt comprising at least one of CO₃, NO₂ and NO₃. It is preferred that said metal salt comprise a Group IA or IIA metal salt. It is most preferred that at least a portion of the aqueous phase from said Separator(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one source of Gas Flow, at least one Scrubber having a source of water flow and at least one Greenhouse and/or ABR form a manufacturing plant and/or process flow path, wherein said source(s) of Gas Flow is upstream of said Scrubber(s) and said Scrubber(s) is upstream of said Greenhouse(s) and/or ABR(s), wherein the water in said Scrubber(s) comprises at least one of: a dispersant and a dispersant in combination with a metal salt, and wherein said Greenhouse(s) and/or ABR(s) converts CO₂ into O₂ and plant growth. It is most preferred that said plant growth comprise algae. It is preferred that said metal salt comprise a Group IA or IIA metal salt. It is most preferred that at least a portion of the aqueous phase in said Greenhouse(s) and/or ABR(s) comprise at least one of Thiobacillus and Thiobacillus denitrificanus. It is most preferred that at least a portion of the aqueous phase from said Greenhouse(s) and/or ABR(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one source of Gas flow, at least one Scrubber having a source of water flow, at least one Salt Reactor and at least one Greenhouse and/or ABR form a manufacturing plant and/or process flow path, wherein said source(s) of Gas Flow is upstream of said Scrubber(s), said Scrubber(s) is upstream of said Salt Reactor(s) and/or said Greenhouse(s) and/or ABR(s), wherein the water in said Scrubber(s) comprises at least one of: a dispersant and a dispersant in combination with a metal salt, wherein said Salt Reactor(s) forms from the reaction of an aqueous solution with metal salt a metal-CO₃ salt, and wherein said Greenhouse(s) and/or ABR(s) converts CO₂ into O₂ and plant growth. It is most preferred that said plant growth comprise algae. It is preferred that said metal salt comprise a Group IA or IIA metal salt. It is most preferred that at least a portion of the aqueous phase in said Greenhouse(s) and/or ABR(s) comprise at least one of Thiobacillus and Thiobacillus denitrificanus. It is most preferred that at least a portion of the aqueous phase from said Greenhouse(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one source of Gas Flow, at least one Scrubber having a source of water flow and at least one Greenhouse and/or ABR form a manufacturing plant and/or process flow path, wherein said source(s) of Gas Flow is upstream of said Scrubber(s) and said Scrubber(s) is upstream of said Greenhouse(s) and/or ABR(s), wherein the water in said Scrubber(s) comprises at least one of: a dispersant and a dispersant in combination with a metal salt, wherein said Greenhouse(s) and/or ABR(s) an acid converts metal-CO₃ from said Scrubber into a metal salt and CO₂ gas, and wherein said Greenhouse(s) and/or ABR(s) converts at least one selected from the list consisting of: said CO₂ gas into O₂ plant growth. It is most preferred that said plant growth comprise algae. It is preferred that said metal salt comprise a Group IA or IIA metal salt. It is most preferred that said acid comprise sulfuric acid. It is most preferred that at least a portion of the aqueous phase in said Greenhouse(s) and/or ABR(s) comprise at least one of Thiobacillus and Thiobacillus denitrificanus. It is most preferred that at least a portion of the aqueous phase from said Greenhouse(s) and/or ABR(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one source of Gas Flow, at least one Scrubber having a source of water flow, at least one Salt Reactor and at least one Greenhouse and/or ABR form a manufacturing plant and/or process flow path, wherein said Source(s) of CO_{X} is upstream of said Scrubber(s) and said Scrubber(s) is upstream of said Greenhouse(s) and/or ABR(s), wherein the water in said Scrubber(s) comprises at least one of a dispersant and a dispersant in combination with a metal salt, wherein said Salt Reactor(s) forms from the reaction of an aqueous solution with metal salt a metal-CO₃ salt, wherein said Greenhouse(s) and/or ABR(s) an acid converts metal-CO₃ from said Scrubber into a metal salt and CO₂ gas, and wherein said Greenhouse(s) and/or ABR(s) converts at least one selected from the list consisting of said CO₂ gas into O₂ plant growth. It is most preferred that said plant growth comprise algae. It is preferred that said metal salt comprise a Group IA or IIA metal salt. It is most preferred that said acid comprise sulfuric acid. It is most preferred that at least a portion of the aqueous phase in said Greenhouse(s) and/or ABR(s) comprise at least one of Thiobacillus and Thiobacillus denitrificanus. It is most preferred that at least a portion of the aqueous phase from said Greenhouse(s) and/or ABR(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise least one Source of CO_{X} gas flow, at least one Scrubber having a source of water flow, at least one Separator, at least one Mode of Solids Transportation and at least Greenhouse and/or ABR form a manufacturing plant and/or process flow path, wherein said Source(s) of CO_{X} is upstream of said Scrubber(s), said Scrubber(s) is upstream of said Separator(s), said Mode of Solids Transport is upstream of said Greenhouse(s) and/or ABR(s), wherein the water in said Scrubber(s) comprises at least one of a dispersant and a dispersant in combination with a metal salt, wherein said Mode(s) of Solids Transport transports at least one metal salt comprising a metal-CO₃ from said Separator(s) to said Greenhouse(s) and/or ABR(s), wherein an acid converts metal-CO₃ from said Scrubber(s) into a metal salt and CO₂ gas, and wherein said Greenhouse(s) and/or ABR(s) converts said CO₂ gas into O₂ plant growth. It is most preferred that said plant growth comprise algae. It is preferred that said metal salt comprise a Group IA or IIA metal salt. It is most preferred that said acid comprise sulfuric acid. It is most preferred that at least a portion of the aqueous phase in said Greenhouse(s) and/or ABR(s) comprise at least one of Thiobacillus and Thiobacillus denitrificanus. It is most preferred that at least a portion of the aqueous phase from said Greenhouse(s) and/or ABR(s) and/or said Separator(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise least one Source of CO_{X} gas flow, at least one Scrubber having a source of water flow, at least one Salt Reactor, at least one Separator, at least one Mode of Solids Transportation and at least Greenhouse and/or ABR form a manufacturing plant and/or process flow path, wherein said Source(s) of CO_{X} is upstream of said Scrubber(s), said Scrubber(s) is upstream of said Salt Reactors and/or said Separator(s) said Mode of Solids Transport is upstream of said Greenhouse(s) and/or ABR(s), wherein the water in said Scrubber(s) comprises at least one of: a dispersant and a dispersant in combination with a metal salt, wherein said Salt Reactor(s) forms from the reaction of an aqueous solution with metal salt a metal-CO₃ salt, wherein said Mode(s) of Solids Transport transports at least one metal salt comprising a metal-CO₃ from said Separator(s) to said Greenhouse(s) and/or ABR(s), wherein an acid converts metal-CO₃ from said Scrubber(s) into a metal salt and CO₂ gas, and wherein said Greenhouse(s) and/or ABR(s) converts said CO₂ gas into O₂ plant growth. It is most preferred that said plant growth comprise algae. It is preferred that said metal salt comprise a Group IA or IIA metal salt. It is most preferred that said acid comprise sulfuric acid. It is most preferred that at least a portion of the aqueous phase in said Greenhouse(s) and/or ABR(s) comprise at least one of Thiobacillus and Thiobacillus denitrificanus. It is most preferred that at least a portion of the aqueous phase from said Greenhouse(s) and/or ABR(s) and/or said Separator(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow and at least one Scrubber having a source of water flow form a manufacturing plant and/or process flow path, wherein said Combustion Source(s) is upstream of said Scrubber(s) and wherein the water in said Scrubber(s) comprises at least one of: a dispersant and a dispersant in combination with a metal salt. It is preferred that said metal salt comprise a Group IA or IIA metal salt. It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Catalysis Unit, and at least one Scrubber having a source of water flow form a manufacturing plant and/or process flow path, wherein said combustion source(s) is upstream of said Catalysis Unit(s), said Catalysis Unit(s) is upstream of said Scrubber(s), wherein the water in said Scrubber(s) comprises at least one of: a dispersant and a dispersant in combination with a metal salt and wherein said Catalysis Unit(s) comprise at least one of Platinum and Rhodium. It is preferred that said metal salt comprise a Group IA or IIA metal salt. It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Scrubber having a source of water flow and at least one Separator form a manufacturing plant and/or process flow path, wherein said combustion source(s) is upstream of said Scrubber(s) and said Scrubber(s) is upstream of said Separator(s), wherein the water in said Scrubber(s) comprises at least one of: a dispersant and a dispersant in combination with a metal salt, and wherein the solid phase from said Separator(s) comprises a metal salt comprising at least one of CO₃, NO₂ and NO₃. It is preferred that said metal salt comprise a Group IA or IIA metal salt. It is most preferred that at least a portion of the aqueous phase from said Separator(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Catalysis Unit, at least one Scrubber having a source of water flow and at least one Separator form a manufacturing plant and/or process flow path, wherein said combustion source(s) is upstream of said Catalysis Unit(s), said Catalysis Unit(s) is upstream of said Scrubber(s) and said Scrubber(s) is upstream of said Separator(s), wherein said Catalysis Unit(s) comprise at least one of Platinum and Rhodium, wherein the water in said Scrubber(s) comprises at least one of: a dispersant and a dispersant in combination with a metal salt, and wherein the solid phase from said Separator(s) comprises a metal salt comprising at least one of CO₃, NO₂ and NO₃. It is preferred that said metal salt comprise a Group IA or IIA metal salt. It is most preferred that at least a portion of the aqueous phase from said Separator(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Scrubber having a source of water flow, at least one Salt Reactor and at least one Separator form a manufacturing plant and/or process flow path, wherein said Combustion Source(s) is upstream of said Catalysis Unit(s), said Scrubber(s) is upstream of said Salt Reactor(s) and/or said Separator(s), wherein the water in said Scrubber(s) comprises at least one of: a dispersant and a dispersant in combination with a metal salt, wherein said Salt Reactor(s) forms from the reaction of an aqueous solution with metal salt a metal-CO₃ salt and wherein the solid phase from said Separator(s) comprises a metal salt comprising at least one of CO₃, NO₂ and NO₃. It is preferred that said metal salt comprise a Group IA or IIA metal salt. It is most preferred that at least a portion of the aqueous phase from said Separator(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Catalysis Unit, at least one Scrubber having a source of water flow, at least one Salt Reactor and at least one Separator form a manufacturing plant and/or process flow path, wherein said Combustion Source(s) is upstream of said Catalysis Unit(s), said Catalysis Unit(s) are upstream of said Scrubber(s) and said Scrubber(s) is upstream of said Salt Reactor(s) and/or said Separator(s), wherein the water in said Scrubber(s) comprises at least one of: a dispersant and a dispersant in combination with a metal salt, wherein said Catalysis Unit(s) comprise at least one of Platinum and Rhodium, wherein said Salt Reactor(s) forms from the reaction of an aqueous solution with metal salt a metal-CO₃ salt and wherein the solid phase from said Separator(s) comprises a metal salt comprising at least one of CO₃, NO₂ and NO₃. It is preferred that said metal salt comprise a Group IA or IIA metal salt. It is most preferred that at least a portion of the aqueous phase from said Separator(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Scrubber having a source of water flow, at least one Separator and at least one Facultative Bio-Reactor form a manufacturing plant and/or process flow path, wherein said Combustion Source(s) is upstream of said Scrubber(s), said Scrubber(s) is upstream of said Separator(s) and said Separator(s) is upstream of said Facultative Bio-Reactor(s), wherein the water in said Scrubber(s) comprises at least one of: a dispersant and a dispersant in combination with a metal salt, wherein the solid phase from said Separator(s) comprises a metal salt comprising at least one of CO₃, NO₂ and NO₃, and wherein said Facultative Bio-Reactor(s) converts at least a portion of the NO₂ and/or NO₃ in the aqueous phase from said Separator(s) into N₂. It is preferred that said metal salt comprise a Group IA or IIA metal salt. It is most preferred that at least a portion of the aqueous phase in said Facultative Bio-Reactor comprise at least one of Thiobacillus and Thiobacillus denitrificanus. It is most preferred that at least a portion of the aqueous phase from said Separator(s) and/or said Facultative Bio-Reactor(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion source having a gas flow, at least one Catalysis Unit, cat least one Scrubber having a source of water flow, at least one Separator and at least one Facultative Bio-Reactor form a manufacturing plant and/or process flow path, wherein said Combustion Source(s) is upstream of said Catalysis Unit(s), said Catalysis Unit(s) is upstream of said Scrubber(s), said Scrubber(s) is upstream of said Separator(s) and said Separator(s) is upstream of said Facultative Bio-Reactor(s), wherein said Catalysis Units comprise at least one of Platinum and Rhodium, wherein the water in said Scrubber(s) comprises at least one of: a dispersant and a dispersant in combination with a metal salt, wherein the solid phase from said Separator(s) comprises a metal salt comprising at least one of CO₃, NO₂ and NO₃, and wherein said Facultative Bio-Reactor(s) converts at least a portion of the NO₂ and/or NO₃ in the aqueous phase from said Separator(s) into N₂. It is preferred that said metal salt comprise a Group IA or IIA metal salt. It is most preferred that at least a portion of the aqueous phase in said Facultative Bio-Reactor comprise at least one of Thiobacillus and Thiobacillus denitrificanus. It is most preferred that at least a portion of the aqueous phase from said Separator(s) and/or said Facultative Bio-Reactor(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Scrubber having a source of water flow, at least one Salt Reactor and at least one Greenhouse and/or ABR form a manufacturing plant and/or process flow path, wherein said Combustion Source(s) is upstream of said Scrubber(s) and said Scrubber(s) is upstream of said Salt Reactor(s) and/or said Greenhouse(s) and/or ABR(s), wherein the water in said Scrubber(s) comprises at least one of: a dispersant and a dispersant in combination with a metal salt, wherein said Salt Reactor(s) forms from the reaction of an aqueous solution with metal salt a metal-CO₃ salt and wherein said Greenhouse(s) and/or ABR(s) converts CO₂ into O₂ and plant growth. It is most preferred that said plant growth comprise algae. It is preferred that said metal salt comprise a Group IA or IIA metal salt It is most preferred that at least a portion of the aqueous phase in said Greenhouse(s) and/or ABR(s) comprise at least one of Thiobacillus and Thiobacillus denitrificanus. It is most preferred that at least a portion of the aqueous phase from said Greenhouse(s) and/or ABR(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Catalysis Unit, at least one Scrubber having a source of water flow, at least one Salt Reactor and at least one Greenhouse and/or ABR form a manufacturing plant and/or process flow path, wherein said Combustion Source(s) is upstream of said Catalysis Units(s), said Catalysis Unit(s) is upstream of said Scrubber(s) and said Scrubber(s) is upstream of said Salt Reactor(s) and/or said Greenhouse(s) and/or ABR(s), wherein said Catalysis Units comprise at least one of Platinum and Rhodium, wherein the water in said Scrubber(s) comprises at least one of: a dispersant and a dispersant in combination with a metal salt, wherein said Salt Reactor(s) forms from the reaction of an aqueous solution with metal salt a metal-CO₃ salt and wherein said Greenhouse(s) and/or ABR(s) converts CO₂ into O₂ and plant growth. It is most preferred that said plant growth comprise algae. It is preferred that said metal salt comprise a Group IA or IIA metal salt. It is most preferred that at least a portion of the aqueous phase in said Greenhouse(s) and/or ABR(s) comprise at least one of Thiobacillus and Thiobacillus denitrificanus. It is most preferred that at least a portion of the aqueous phase from said Greenhouse(s) and/or ABR(s) flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Scrubber having a source of water flow, at least one Facultative Bio-Reactor and at least one Greenhouse and/or ABR form a manufacturing plant and/or process flow path, wherein said Combustion source(s) is upstream of said Scrubber(s), said Scrubber(s) is upstream of said Separator(s), said Separator(s) is upstream of said Facultative Bio-Reactor(s) and said Greenhouse(s) and/or ABR(s), wherein the water in said Scrubber(s) comprises at least one of: a dispersant and a dispersant in combination with a metal salt, wherein the solid phase from said Separator(s) comprises a metal salt comprising at least one of CO NO₂ and NO₃, wherein at least a portion of the aqueous phase from said Separator(s) flows to said Facultative Bio-Reactor(s), wherein said Facultative Bio-Reactor(s) converts at least a portion of the NO₂ and/or NO₃ in the aqueous phase from said Separator(s) into N₂, and wherein said Greenhouse(s) and/or ABR(s) converts CO₂ into O₂ and plant growth. It is most preferred that said plant growth comprise algae. It is preferred that said metal salt comprise a Group IA or IIA metal salt. It is most preferred that at least a portion of the aqueous phase in said Greenhouse(s) and/or ABR(s) and/or said Facultative Bio-Reactor(s) comprise at least one of Thiobacillus and Thiobacillus denitrificanus. It is most preferred that at least a portion of the aqueous phase from at least one selected from the list consisting of said Separator(s), said Facultative Bio-Reactor(s), said Greenhouse(s) and/or ABR(s), and any combination therein, flow back to at least one of said Scrubber(s). It is most preferred that at least one unit add said dispersant and/or said metal salt to said water in said Scrubber(s) and/or to the water prior to entering said Scrubber(s).

It is a preferred embodiment that an apparatus comprise at least one Combustion Source having a gas flow, at least one Catalysis Unit, at least one Scrubber having a source of water flow, at least one Facultative Bio-Reactor and at least one Greenhouse and/or ABR form a manufacturing plant and/or process flow path, wherein said Combustion Source(s) is upstream of said Catalysis Unit(s), said Catalysis Unit(s) is upstream of said Scrubber(s), said Scrubber(s) is upstream of said Separator(s), said Separator(s) is upstream of said Facultative Bio-Reactor(s) and said Greenhouse(s) and/or ABR(s), wherein said Catalysis Units comprise at least one of Platinum and Rhodium, wherein the water in said Scrubber(s) comprises at least one of: a dispersant and a dispersant in combination with a metal salt, wherein the solid phase from said Separator(s) comprises a metal salt comprising at least one of CO₃, NO₂ and NO₃, wherein at least a portion of the aqueous phase from said Separator(s) flows to said Facultative Bio-Reactor(s), wherein said Facultative Bio-Reactor(s) converts at least a portion of the NO₂ and/or NO₃ in the aqueous phase from said Separator(s) into N₂, and wherein said Greenhouse(s) and/or ABR(s) converts CO₂ into O₂ and plant growth. It is most preferred that said plant growth comprise algae. It is preferred that said metal salt comprise a Group IA or IIA metal salt. It is most preferred that at least a portion of the aqueous phase in said Greenhouse(s) and/or ABR(s) and/or said Facultative Bio-Reactor(s) comprise at least one of Thiobacillus and Thiobacillus denitrificanus. It is most preferred that at least a portion of the aqueous phase from at least one selected from the list consisting of: said Separator(s), said Facultative Bio-Reactor(s), said Greenhouse(s) and/or ABR(s), and any combination therein, flow back to at least one of said Scrubber(s). It is most preferred that at least one unit add said dispersant and/or said metal salt to said water in said Scrubber(s) and/or to the water prior to entering said Scrubber(s).

It is a preferred embodiment that apparatus comprise at least one Combustion Source having a gas flow, at least one Scrubber having a source of water flow, at least one Separator, at least one Facultative Bio-Reactor and at least one Greenhouse and/or ABR form a manufacturing plant and/or process flow path, wherein said Combustion Source(s) is upstream of said Scrubber(s), said Scrubber(s) is upstream of said Separator(s), said Separator(s) is upstream of said Facultative Bio-Reactor(s) and said Greenhouse(s) and/or ABR(s), wherein the water in said Scrubber(s) comprises at least one of: a dispersant and a dispersant in combination with a metal salt, wherein the solid phase from said Separator(s) comprises a metal salt comprising at least one selected from the list consisting of: CO₃, NO₂, NO₃ and any combination therein, wherein at least a portion of the aqueous phase from said Separator(s) flows to said Facultative Bio-Reactor(s), wherein said Facultative Bio-Reactor(s) converts at least a portion of the NO₂ and/or NO₃ in the aqueous phase from said Separator(s) into N₂, and wherein said Greenhouse(s) and/or ABR(s) converts CO₂ into O₂ and plant growth. It is most preferred that said plant growth comprise algae. It is preferred that said metal salt comprise a Group IA or IIA metal salt. It is most preferred that at least a portion of the aqueous phase in said Greenhouse(s) and/or ABR(s) and/or said Facultative Bio-Reactor(s) comprise at least one of Thiobacillus and Thiobacillus denitrificanus. It is most preferred that at least a portion of the aqueous phase from at least one selected from the list consisting of: said Separator(s), said Facultative Bio-Reactor(s), said Greenhouse(s) and/or ABR(s), and any combination therein, flow back to at least one of said Scrubber(s). It is most preferred that at least one unit add said dispersant and/or said metal salt to said water in said Scrubber(s) and/or to the water prior to entering said Scrubber(s). It is most preferred that said solid phase from said Separator(s) have a Mode of Transport to said Greenhouse(s) and/or ABR(s).

It is a preferred embodiment that an apparatus comprise at least one Combustion Source having a gas flow, at least one Catalysis Unit, at least one Scrubber having a source of water flow, at least one Separator, at least one Facultative Bio-Reactor and at least one Greenhouse and/or ABR form a manufacturing plant and/or process flow path, wherein said Combustion Source(s) is upstream of said Catalysis Unit(s), said Catalysis Unit(s) is upstream of said Scrubber(s), said Scrubber(s) is upstream of said Separator(s), said Separator(s) is upstream of said Facultative Bio-Reactor(s) and said Greenhouse(s) and/or ABR(s), wherein said Catalysis Units comprise at least one of Platinum and Rhodium, wherein the water in said Scrubber(s) comprises at least one of: a dispersant and a dispersant in combination with a metal salt, wherein the solid phase from said Separator(s) comprises a metal salt comprising at least one selected from the list consisting of: CO₃, NO₂, NO₃ and any combination therein, wherein at least a portion of the aqueous phase from said Separator(s) flows to said Facultative Bio-Reactor(s), wherein said Facultative Bio-Reactor(s) converts at least a portion of the NO₂ and/or NO₃ in the aqueous phase from said Separator(s) into N₂, and wherein said Greenhouse(s) and/or ABR(s) converts CO₂ into O₂ and plant growth. It is most preferred that said plant growth comprise algae. It is preferred that said metal salt comprise a Group IA or IIA metal salt It is most preferred that at least a portion of the aqueous phase in said Greenhouse(s) and/or ABR(s) and/or said Facultative Bio-Reactor(s) comprise at least one of Thiobacillus and Thiobacillus denitrificanus. It is most preferred that at least a portion of the aqueous phase from at least one selected from the list consisting of said Separator(s), said Facultative Bio-Reactor(s), said Greenhouse(s) and/or ABR(s), and any combination therein, flow back to at least one of said Scrubber(s). It is most preferred that at least one unit add said dispersant and/or said metal salt to said water in said Scrubber(s) and/or to the water prior to entering said Scrubber(s). It is most preferred that said solid phase from said Separator(s) have a Mode of Transport to said Greenhouse(s) and/or ABR(s).

It is a preferred embodiment that an apparatus comprise at least one Combustion Source having a gas flow, at least one Scrubber having a source of water flow, at least one Salt Reactor, at least one Separator, at least one Facultative Bio-Reactor and at least one Greenhouse and/or ABR form a manufacturing plant and/or process flow path, wherein said Combustion Source(s) is upstream of said Scrubber(s), said Scrubber(s) is upstream of said Salt Reactor(s) and/or said Separator(s), said Salt Reactor(s) is upstream of said Separator(s), said Separator(s) is upstream of said Facultative Bio-Reactor(s) and said Greenhouse(s) and/or ABR(s), wherein the water in said Scrubber(s) comprises at least one of: a dispersant and a dispersant in combination with a metal salt, wherein said Salt Reactor(s) react a metal salt with the aqueous phase from said Scrubber(s) to form a metal salt comprising at least one selected from the list consisting of: CO₃, NO₂, NO₃ and any combination therein, wherein the solid phase from said Separator(s) comprises a metal salt comprising at least one selected from the list consisting of: CO₃, NO₂, NO₃ and any combination therein, wherein at least a portion of the aqueous phase from said Separator(s) flows to said Facultative Bio-Reactor(s), wherein said Facultative Bio-Reactor(s) converts at least a portion of the NO₂ and/or NO₃ in the aqueous phase from said Separator(s) into N₂, and wherein said Greenhouse(s) and/or ABR(s) converts CO₂ into O₂ and plant growth. It is most preferred that said plant growth comprise algae. It is preferred that said metal salt comprise a Group IA or IIA metal salt. It is most preferred that at least a portion of the aqueous phase in said Greenhouse and/or said Facultative Bio-Reactor comprise at least one of Thiobacillus and Thiobacillus denitrificanus. It is most preferred that at least a portion of the aqueous phase from at least one selected from the list consisting of: said Separator(s), said Facultative Bio-Reactor(s), said Greenhouse(s) and/or ABR(s), and any combination therein, flow back to at least one of said Scrubber(s). It is most preferred that at least one unit add said dispersant and/or said metal salt to said water in said Scrubber(s) and/or to the water prior to entering said Scrubber(s). It is most preferred that said solid phase from said Separator(s) have a Mode of Transport to said Greenhouse(s) and/or ABR(s).

It is a preferred embodiment that an apparatus comprise at least one Combustion Source having a gas flow, at least one Catalysis Unit, at least one Scrubber having a source of water flow, at least one Salt Reactor, at least one Separator, at least one Facultative Bio-Reactor and at least one Greenhouse and/or ABR form a manufacturing plant and/or process flow path, wherein said Combustion Source(s) is upstream of said Catalysis Unit(s), said Catalysis Unit(s) is upstream of said Scrubber(s), said Scrubber(s) is upstream of said Salt Reactor(s) and/or said Separator(s), said Salt Reactor(s) is upstream of said Separator(s), said Separator(s) is upstream of said Facultative Bio-Reactor(s) and said Greenhouse(s) and/or ABR(s), wherein said Catalysis Units comprise at least one of Platinum and Rhodium, wherein the water in said Scrubber(s) comprises at least one of: a dispersant and a dispersant in combination with a metal salt, wherein said Salt Reactor(s) react a metal salt with the aqueous phase from said Scrubber(s) to form a metal salt comprising at least one selected from the list consisting of: CO₃, NO₂, NO₃ and any combination therein, wherein the solid phase from said Separator(s) comprises a metal salt comprising at least one selected from the list consisting of: CO₃, NO₂, NO₃ and any combination therein, wherein at least a portion of the aqueous phase from said Separator(s) flows to said Facultative Bio-Reactor(s), wherein said Facultative Bio-Reactor(s) converts at least a portion of the NO₂ and/or NO₃ in the aqueous phase from said Separator(s) into N₂, and wherein said Greenhouse(s) and/or ABR(s) converts CO₂ into O₂ and plant growth. It is most preferred that said plant growth comprise algae. It is preferred that said metal salt comprise a Group IA or IIA metal salt. It is most preferred that at least a portion of the aqueous phase in said Greenhouse(s) and/or ABR(s) and/or said Facultative Bio-Reactor(s) comprise at least one of Thiobacillus and Thiobacillus denitrificanus. It is most preferred that at least a portion of the aqueous phase from at least one selected from the list consisting of said Separator(s), said Facultative Bio-Reactor(s), said Greenhouse(s) and/or ABR(s), and any combination therein, flow back to at least one of said Scrubber(s). It is most preferred that at least one unit add said dispersant and/or said metal salt to said water in said Scrubber(s) and/or to the water prior to entering said Scrubber(s). It is most preferred that said solid phase from said Separator(s) have a Mode of Transport to said Greenhouse(s) and/or ABR(s).

It is a preferred embodiment for an apparatus or a manufacturing flow path comprising a Gas flow, wherein the Gas flow is upstream of at least one ABR unit comprising an aqueous solution, wherein the ABR unit(s) converts at least a portion of the CO_{X} into O₂ and biomass, and wherein the ABR unit(s) comprises at least one selected from the group consisting of: a number of the ABR unit(s) arranged side-by-side in a circular pattern forming an ABR Cluster Unit, a number of annular shaped ABR(s) comprising a tube within a tube, wherein the ABR(s) comprise the annular portion between the radii of outside an the inside tube and the photons enter each ABR from the center tube, a tube dispersing photons into the ABR unit(s), the ABR unit(s) comprise contact with photons, wherein the transference of photons to said ABR(s) comprises at least one of a tube and a fiber optic cable, the ABR unit(s) comprise insulation, the ABR unit(s) comprise a tubular shape comprising a tube dispersing the gas into the ABR(s), the ABR(s) comprise a CSTR comprising at least one tube dispersing photons to each ABR(s), the ABR unit(s) comprise a membrane for dispersing the gas into the ABR(s), and any combination therein.

It is preferred that the Gas flow(s) comprises a combustion source. It is preferred that the Gas flow(s) comprises a unit cooling the Gas flow(s).

It is preferred that at least one unit add a dispersant to the aqueous solution.

It is preferred that at least one unit add at least one nutrient to the aqueous solution.

It is preferred that at least one unit add to the aqueous solution at least one selected from the group consisting of hydroxide, bi-carbonate, magnesium, and any combination therein.

It is preferred that at least one unit add to the aqueous solution, either upstream of or within said ABR(s), a Group IA or IIA metal salt.

It is preferred that at least one unit heat or cool the aqueous solution.

It is preferred that at least one unit downstream of the ABR unit(s) or ABR Cluster unit perform gas/liquid separation of the effluent aqueous solution from the ABR unit(s) or ABR Cluster(s) or CSTR ABR(s). It is preferred that the liquid from the gas/liquid separation return to the aqueous solution. It is preferred that the effluent from the ABR unit(s) or ABR Cluster(s) or CSTR ABR(s) at least partially bypass gas/liquid separation, wherein the effluent aqueous solution is returned to the aqueous solution. It is preferred that the ABR unit(s) or ABR Cluster(s) or ABR CSTR(s) produce O₂ and a unit separates the O₂ from the gas. It is preferred that when the ABR(s), ABR unit(s) or ABR Cluster(s) or ABR CSTR(s) produce H₂, a unit downstream of the gas/liquid separation unit a least partially separate H₂ from the gas. It is preferred that the gas separation unit comprises is at least one of: membrane, vacuum swing adsorption, pressure swing adsorption, and cryogenic distillation.

It is a preferred embodiment that at least one ABR unit produce H₂ and at least one ABR unit produce O₂. It is a preferred embodiment that at least one ABR unit produce H₂ and at least one ABR unit produce O₂, wherein at least a portion of the H₂ and at least a portion of the O₂ is used in a unit to provide power to or heat to the ABR(s). It is a preferred embodiment that at least on ABR unit produce H₂ and at least one ABR unit produce O₂, wherein at least a portion of the H₂ and at least a portion of the O₂ is used in a unit to provide power for a unit to perform separation of at least one of O₂ from the gas, and H₂ from the gas.

It is preferred that at least one unit combust at least a portion of at least one selected from the list consisting of the: hydrocarbon product of the algae, H₂, at least a portion of the algae itself from within at least on ABR, and any combination therein to generate electrical energy. It is preferred that at least a portion said electricity be used in a unit to produce photons for at least one of the ABR unit(s).

It is a preferred embodiment that the liquid from the gas/liquid separation unit enter an FBR unit, wherein at least one of: NO₂ or NO₃ is converted into N₂, and S_{X} is converted into sulfur within the biomass of sulfur consuming bacteria. It is an embodiment that the liquid from the gas separation unit enter a unit performing liquid/solids separation of the liquid, wherein the liquid is separated into mostly an aqueous portion and mostly a solids portion, and wherein the solids portion comprises algae. It is preferred that at least a portion of the aqueous phase return to the aqueous solution. It is preferred that the solids portion be transferred to a liquid/solids separation unit, wherein the amount of liquid with the algae is reduced in the solids portion.

It is an embodiment that the ABR unit(s) comprises a media.

It is preferred that a unit acidify a metal-CO₃ to produce CO_{X} for the ABR unit(s) or ABR Cluster Unit It is preferred that a unit acidify a metal-CO₃ to produce CO_{X} for the ABR Cluster Unit. It is preferred that a unit acidify a metal-NO₂ or a metal-NO₃ to produce NO_{X} for the ABR unit(s) or the ABR Cluster Unit. It is most preferred that the acid comprise carbonic or sulfuric acid.

It is a preferred embodiment that an apparatus or a manufacturing process flow path comprise at least one Gas flow, at least one FBR and at least one ABR, wherein the Gas flow(s) is upstream of the FBR(s), wherein the FBR(s) is upstream of the ABR(s), and wherein the ABR(s) convert CO₂ into at least one of O₂ and H₂, along with biomass. It is most preferred that at least a portion of the aqueous solution in the ABR(s) comprise at least one specie of algae. It is most preferred that at least a portion of the aqueous phase in the FBR(s) comprise at least one specie of facultative bacteria. It is preferred that at least a portion of the aqueous phase in the FBR(s) comprise at least one specie of sulfide consuming bacteria. It is most preferred that at least a portion of the aqueous phase in the FBR(s) comprise at least one species of the genus Thiobacillus or the species Thiobacillus denitrificans. It is most preferred that at least a portion of the aqueous solution in the ABR(s) comprise at least one species of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one species of the genus Thiobacillus, such as Thiobacillus denitrificans.

It is a preferred embodiment that an apparatus or a manufacturing process flow path comprise at least one Gas flow, at least one FBR and at least one ABR, wherein the Gas flow(s) is upstream of the ABR(s), wherein the SBR(s) is upstream of the FBR(s), and wherein the ABR(s) convert CO₂ into at least one of O₂ and H₂, along with algae. It is most preferred that at least a portion of the aqueous solution in the ABR(s) comprise at least one species of algae. It is most preferred that at least a portion of the aqueous phase in the FBR(s) comprise at least one specie of facultative bacteria. It is preferred that at least a portion of the aqueous phase in the FBR(s) comprise at least one specie of sulfide consuming bacteria. It is most preferred that at least a portion of the aqueous phase in the FBR(s) comprise at least one of the genus Thiobacillus or the species Thiobacillus denitrificans. It is most preferred that at least a portion of the aqueous solution in the ABR(s) comprise at least one specie of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one of the species of the genus Thiobacillus, such as Thiobacillus denitrificans.

It is a preferred embodiment that an apparatus or manufacturing process flow path comprises at least one Gas flow and at least one Scrubber having a source of water flow, wherein the Gas flow(s) is upstream of the Scrubber(s) and wherein the aqueous solution in the Scrubber(s) comprises at least one of: a dispersant and a metal salt. It is preferred that the metal salt comprise a Group IA or IIA metal. It is most preferred that at least one unit add at least one of the dispersant and the metal salt to the aqueous solution in the Scrubber(s) or to the water prior to the Scrubber(s).

It is a preferred embodiment that an apparatus or manufacturing process flow path comprise at least one Gas flow, at least one Scrubber having a source of water flow and at least one ABR, wherein the Gas flow(s) is upstream of the Scrubber(s) and the Scrubber(s) is upstream of the ABR(s), wherein the aqueous solution in the Scrubber(s) comprises at least one of a dispersant and a metal salt, and wherein the ABR(s) convert CO₂ into at least one of O₂ and H₂, along with algae. It is preferred that the metal salt comprise a Group IA or IIA metal. It is most preferred that at least a portion of the aqueous solution in the ABR(s) comprise at least one specie of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one of the species of the genus Thiobacillus, such as the species Thiobacillus denitrificans. It is most preferred that at least a portion of the aqueous solution from the ABR(s) flow back to at least one of the Scrubber(s). It is most preferred that at least one unit add at least one of the dispersant and the metal salt to the aqueous solution in the Scrubber(s) or to the water prior to the Scrubber(s).

It is a preferred embodiment that an apparatus or manufacturing process flow path comprise at least one Gas flow, at least one Scrubber having a source of water flow and at least one ABR, wherein the Gas flow(s) is upstream of the Scrubber(s) and the Scrubber(s) is upstream of the ABR(s), wherein the aqueous solution in the Scrubber(s) comprises at least one of: a dispersant and a metal salt, wherein an acid converts metal-CO₃ from the Scrubber(s) into a metal salt and CO₂ gas, and wherein the ABR(s) convert at least a portion of the Gas flow(s) into at least one of O₂ and H₂, along with algae. It is preferred that the metal salt comprise a Group IA or IIA metal. It is most preferred that the acid comprise sulfuric acid. It is most preferred that at least a portion of the aqueous solution in the ABR(s) comprise at least one specie of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one of the species of the genus Thiobacillus, such as the species Thiobacillus denitrificans. It is most preferred that at least a portion of the aqueous solution from the ABR(s) flow back to at least one of the Scrubber(s). It is most preferred that at least one unit add at least one of the dispersant and the metal salt to the aqueous solution in the Scrubber(s) or to the water prior to the Scrubber(s).

It is a preferred embodiment that an apparatus or manufacturing process flow path comprise at least one Gas flow, at least one Scrubber having a source of water flow, at least one separator and at least one ABR, wherein the Gas Flow(s) is upstream of the Scrubber(s) and the Scrubber(s) is upstream of the Separator(s) and the Scrubber(s) and the Separator(s) are upstream of the ABR(s), wherein the aqueous phase in the Scrubber(s) comprises at least one of a dispersant and a metal salt, wherein the solid solution from the Separator(s) comprises a metal salt comprising at least one of CO₃, NO₂ and NO₃, wherein an acid converts metal-CO₃ from the Scrubber(s) into a metal salt and CO₂ gas, and wherein the ABR(s) convert at least a portion of the Gas flow(s) into at least one of O₂ and H₂, along with algae. It is preferred that the metal salt comprise a Group IA or IIA metal. It is most preferred that the acid comprise sulfuric acid. It is most preferred that at least a portion of the aqueous solution in the ABR(s) comprise at least one specie of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one of the species of the genus Thiobacillus, such as the species Thiobacillus denitrificans. It is most preferred that at least a portion of the aqueous solution from the ABR(s) flow back to at least one of the Scrubber(s). It is most preferred that at least one unit add at least one of the dispersant and the metal salt to the aqueous solution in the Scrubber(s) or to the water prior to the Scrubber(s).

It is a preferred embodiment that an apparatus or manufacturing process flow path comprise at least one Gas flow, at least one Scrubber having a source of water flow, at least one FBR, and at least one ABR, wherein the Gas Flow(s) is upstream of the Scrubber(s) and the Scrubber(s) is upstream of the FBR(s), and the FBR(s) is upstream of the ABR(s), wherein the aqueous solution in the Scrubber(s) comprises at least one of: a dispersant and a metal salt, wherein an acid converts metal-CO₃ from the Scrubber(s) into a metal salt and CO₂ gas, wherein the FBR converts at least one of NO₂ and NO₃ into N₂, and wherein the ABR(s) convert at least a portion of the Gas flow(s) into at least one of O₂ and H₂, along with algae. It is preferred that the metal salt comprise a Group IA or IIA metal. It is most preferred that at least a portion of the aqueous solution in the ABR(s) comprise at least one specie of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one of the species of the genus Thiobacillus, such as the species Thiobacillus denitrificans. It is most preferred that at least a portion of the aqueous phase in the FBR(s) comprise at least one specie of facultative bacteria. It is most preferred that at least a portion of the aqueous phase in the FBR(s) comprise at least one of the species of the genus Thiobacillus, such as the species Thiobacillus denitrificans. It is most preferred that at least a portion of the aqueous solution from the ABR(s) flow back to at least one of the Scrubber(s). It is most preferred that at least one unit add at least one of the dispersant and the metal salt to the aqueous solution in the Scrubber(s) or to the water prior to the Scrubber(s).

It is a preferred embodiment that an apparatus or manufacturing process flow path comprise at least one Gas flow, at least one Scrubber having a source of water flow, at least one FBR, and at least one ABR, wherein the Gas Flow(s) is upstream of the Scrubber(s) and the Scrubber(s) is upstream of the ABR(s), and the ABR(s) is upstream of the FBR(s), wherein the aqueous solution in the Scrubber(s) comprises at least one of a dispersant and a metal salt, wherein an acid converts metal-CO₃ from the Scrubber(s) into a metal salt and CO₂ gas, wherein the FBR converts at least one of NO₂ and NO₃ into N₂, and wherein the ABR(s) convert at least a portion of the Gas flow(s) into at least one of O₂ and H₂, along with algae. It is preferred that the metal salt comprise a Group IA or IIA metal. It is most preferred that at least a portion of the aqueous solution in the ABR(s) comprise at least one specie of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one of the species of the genus Thiobacillus, such as the species Thiobacillus denitrificans. It is most preferred that at least a portion of the aqueous phase in the FBR(s) comprise at least one specie of facultative bacteria. It is most preferred that at least a portion of the aqueous phase in the FBR(s) comprise at least one of the species of the genus Thiobacillus, such as the species Thiobacillus denitrificans. It is most preferred that at least a portion of the aqueous solution from the ABR(s) flow back to at least one of the Scrubber(s). It is most preferred that at least one unit add at least one of the dispersant and the metal salt to the aqueous solution in the Scrubber(s) or to the water prior to the Scrubber(s).

It is a preferred embodiment that an apparatus or manufacturing process flow path comprise at least one Gas flow, at least one Scrubber having a source of water flow, at least one Separator, at least one FBR, and at least one ABR, wherein the Gas Flow(s) is upstream of the Scrubber(s) and the Scrubber(s) is upstream of the Separator(s), the Scrubber(s) and the Separator(s) are upstream of the ABR(s), and the FBR(s) is upstream of the ABR(s), wherein the aqueous solution in the Scrubber(s) comprises at least one of: a dispersant and a metal salt, wherein the solids from the Separator(s) comprises a metal salt comprising at least one of CO₃, NO₂ and NO₃, wherein an acid converts metal-CO₃ from the Scrubber(s) into a metal salt and CO₂ gas, wherein the FBR converts at least one of NO₂ and NO₃ into N₂, and wherein the ABR(s) convert at least a portion of the Gas flow(s) into at least one of O₂ and H₂, along with algae. It is preferred that the metal salt comprise a Group IA or IIA metal. It is most preferred that the acid comprise sulfuric acid. It is most preferred that at least a portion of the aqueous solution in the ABR(s) comprise at least one specie of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one of the genus Thiobacillus and the specie Thiobacillus denitrificans. It is most preferred that at least a portion of the aqueous solution from the ABR(s) flow back to at least one of the Scrubber(s). It is most preferred that at least one unit add at least one of the dispersant and the metal salt to the aqueous solution in the Scrubber(s) or to the water prior to the Scrubber(s).

It is a preferred embodiment that an apparatus or manufacturing process flow path comprise at least one Gas flow, at least one Scrubber having a source of water flow, at least one Separator, at least one FBR, and at least one ABR, wherein the Gas Flow(s) is upstream of the Scrubber(s) and the Scrubber(s) is upstream of the Separator(s), the Scrubber(s) and the Separator(s) are upstream of the ABR(s), and the ABR(s) is upstream of the FBR(s), wherein the aqueous solution in the Scrubber(s) comprises at least one of: a dispersant and a metal salt, wherein the solids from the Separator(s) comprises a metal salt comprising at least one of CO₃, NO₂ and NO₃, wherein an acid converts metal-CO₃ from the Scrubber(s) into a metal salt and CO₂ gas, wherein the FBR converts at least one of NO₂ and NO₃ into N₂, and wherein the ABR(s) convert at least a portion of the Gas flow(s) into at least one of O₂ and H₂, along with algae. It is preferred that the metal salt comprise a Group IA or IIA metal. It is most preferred that the acid comprise sulfuric acid. It is most preferred that at least a portion of the aqueous solution in the ABR(s) comprise at least one specie of facultative bacteria. It is most preferred that at least a portion of the aqueous solution in the ABR(s) comprise at least one specie of heterotrophic bacteria. It is preferred that at least a portion of the aqueous solution in the ABR(s) comprise at least one specie of sulfide consuming bacteria. It is most preferred that at least a portion of the aqueous solution in the ABR(s) comprise at least one of the genus Thiobacillus and the specie Thiobacillus denitrificans. It is most preferred that at least a portion of the aqueous solution from the ABR(s) flow back to at least one of the Scrubber(s). It is most preferred that at least one unit add at least one of the dispersant and the metal salt to the aqueous solution in the Scrubber(s) or to the water prior to the Scrubber(s).

Certain objects are set forth above and made apparent from the foregoing description. However, since certain changes may be made in the above description without departing from the scope of the invention, it is intended that all matters contained in the foregoing description shall be interpreted as illustrative only of the principles of the invention and not in a limiting sense. With respect to the above description, it is to be realized that any descriptions, drawings and examples deemed readily apparent and obvious to one skilled in the art and all equivalent relationships to those described in the specification are intended to be encompassed by the present invention.

Further, since numerous modifications and changes will readily occur to those skilled in the art, it is not desired to limit the invention to the exact construction and operation shown and described, and accordingly, all suitable modifications and equivalents may be resorted to, falling within the scope of the invention. It is also to be understood that the following claims are intended to cover all of the generic and specific features of the invention herein described, and all statements of the scope of the invention, which, as a matter of language, might be said to fall in between.

We claim:

1. A method of converting a gas comprising CO_{X} into biomass, the method comprising:
contacting the gas with algae in an aqueous solution in at least one ABR, wherein
the ABR(s) converts at least a portion of the CO_{X} into biomass, wherein
the ABR(s) comprises at least one selected from the group consisting of:
a number of the ABR(s) arranged side-by-side in a circular pattern forming an ABR Cluster,
a number of annular shaped ABR(s) comprising a tube within a tube, wherein the ABR(s) comprise the annular portion between the radii of outside an the inside tube and the photons enter each ABR from the center tube,
at least one photon tube dispersing photons into each ABR(s),
the ABR(s) aqueous solution comprises contact with photons, wherein the transference of photons to said ABR(s) comprises at least one of a tube and a fiber optic cable,
the ABR(s) comprise insulation,
the ABR(s) comprise a tubular shape comprising a gas tube dispersing the gas into the ABR(s),
the ABR(s) comprise a continuous stirred tank reactor comprising at least one tube dispersing photons into each ABR(s),
the ABR(s) comprise a membrane for dispersing the gas into the ABR(s), and
any combination therein.

2. The method of claim 1, wherein said gas further comprises NO_{X}, wherein
said ABR converts at least a portion of at least one of NO₂ and NO₃ into algae.

3. The method of claim 1, wherein said gas is from a combustion source.

4. The method of claim 1, wherein O₂ is produced.

5. The method of claim 1, wherein said aqueous solution comprises a dispersant.

6. The method of claim 5, wherein said dispersant comprises a carboxyl or sulfoxy moiety.

7. The method of claim 5, wherein said dispersant comprises at least one selected from the group consisting of: acrylic polymers, acrylic acid, polymers of acrylic acid, methacrylic acid, maleic acid, fumaric acid, itaconic acid, crotonic acid, cinnamic acid, vinyl benzoic acid, any polymers of these acids, and any combination therein.

8. The method of claim 1, wherein said ABR Cluster comprises 6 ABR.

9. The method of claim 1, wherein there is a number of ABR Cluster.

10. The method of claim 1, wherein said photon tube comprises a translucent material, and comprises at least one of:
a one way mirror at one end, the one way mirror allowing photon entrance into said photon tube while reflecting photons from leaving the same end,
a reflective or mirrored surface at the end opposite the end of photon entrance, and
a fiber optic cable.

11. The method of claim 1, wherein said ABR Cluster comprises space between said ABR(s), wherein
the space between said ABR(s) allows photons from said photon tube to pass between said ABR(s), such that
the photons which pass between said ABR(s) are reflected from a reflective mirrored surface onto the side of the ABR(s) which does not face said photon tube.

12. The method of claim 1, wherein said ABR Cluster comprises at least one of
a one way mirror at one end, the one way mirror allowing photon entrance into said ABR Cluster while reflecting photons from leaving the same end,
a reflective or mirrored surface at the end opposite the end of photon entrance, and
a conical shaped reflective or mirrored surface at the end opposite the end of photon entrance.

13. The method of claim 1, wherein said tube or fiber optic cable comprises a reflective or mirrored inside coating.

14. The method of claim 1, wherein said photons are obtained from the Sun by at least one reflective or mirrored surface.

15. The method of claim 14, wherein said reflective or mirrored surface(s) track the location of the Sun.

16. The method of claim 14, wherein said photons from said reflective or mirrored surface(s) are distributed into said tube or said fiber optic cable from a spherical shaped distribution point, and wherein
the spherical shaped distribution point has a reflective or mirrored inside surface.

17. The method of claim 1, wherein said ABR(s) or said ABR Cluster comprises outside of said ABR(s) or ABR Cluster a reflective or mirrored surface to reflect photons emanating from said ABR(s) or ABR Cluster back to said ABR(s) or ABR Cluster.

18. The method of claim 1, wherein said ABR(s) is translucent.

19. The method of claim 1, wherein said ABR(s) comprises at least one of silicon, glass, a conductive material, metal, and any combination therein.

20. The method of claim 19, wherein said ABR(s) comprise a conductive material or a metal comprising a negative electrical charge.

21. The method of claim 1, further comprising vibration or ultrasonics to said ABR(s).

22. The method of claim 1, wherein said ABR(s) comprise at least one algae selected from the group consisting of: Anabaena cylindrical, Bostrychia scorpioides, Botrycoccus braunii, Chaetoceros muelleri, Chlamydomonas moeweesi, Chlamydomonas reinhardtii, Chlorella pyrenoidosa, Chlorella vulgaris, Chlorella vulgaris Beij, Dunaliella bioculata, Dunaliella sauna, Dunaliella tertiolecta, Euglena gracilis, Isochrysis galbana, Isochrysis galbanais micro, Nannochloris sp., Nannochloropsis sauna, Nannochloropsis sauna Nannochloris oculata—N. oculata, N. atomus Butcher, N. maculata Butcher, N. gaditaa Lubian, N. oculata, Neochloris oleoabundans, Nitzschia communis, Parietochloris incise, Phaeodactylum tricornutum, Pleurochrysis carterae, haptophyta, prymnesiophyceae, Porphyridium cruentum, Prymnesium parvum, Scenedesmus dimorphus, Scenedesmus obliquus, Scenedesmus quadricauda, Schenedesmus dimorphus, Spirogyra sp., Spirulina maxima, Spirulina platensis, Spirulina sp., Synechoccus sp., Tetraselmis chui, Tetraselmis chui, Tetraselmis maculate, Tetraselmis suecica, Botrycoccus braunii, Botrycoccus braunii strains, Chlamydomonas reinhardtii, Chlorella vulgaris, Anabaena cylindrical, Chlamydomonas rheinhardii, Chlorella pyrenoidosa, Chlorella vulgaris, Dunaliella bioculata, Dunaliella salina, Euglena gracilis, Porphyridium cruentum, Prymnesium parvum, Scenedesmus dimorphus, Scenedesmus obliquus, Scenedesmus quadricauda, Spirogyra sp., Spirulina maxima, Spirulina platensis, Synechoccus sp., Tetraselmis maculate, and any combination therein.

23. The method of claim 1, wherein said algae comprise selectively cultured algae.

24. The method of claim 1, wherein said algae comprise mutant algae.

25. The method of claim 1, wherein said algae is at least one of non-pathogenic, non-opportunistic, low-virulence factor, and any combination therein.

26. The method of claim 1, wherein said aqueous solution comprises denitrifying bacteria.

27. The method of claim 26, wherein said denitrifying bacteria is at least one of: non-pathogenic, non-opportunistic, low-virulence factor, and any combination therein.

28. The method of claim 1, wherein said aqueous solution comprises sulfur consuming bacteria.

29. The method of claim 1, wherein said aqueous solution comprises at least one selected from the group consisting of: gram-negative bacteria from the beta or gamma subgroup of Proteobacteria, obligate autotrophs, Thioalkalovibrio, strain LMD 96.55, Thioalkalobacter, alkaliphilic heterotrophic bacteria, Pseudomonas strain ChG 3, Rhodococcus erythropolis, Rhodococcus rhodochrous, Rhodococcus sp., Nocardia erythropolis, Nocardia corrolina, Nocardia sp., Pseudomonas putida, Pseudomonas oleovorans, Pseudomonas sp., Arthrobacter globiformis, Arthobacter Nocardia paraffinae, Arthrobacter paraffineus, Arthrobacter citreus, Arthrobacter luteus, Arthrobacter sp., Mycobacterium vaccae JOB, Mycobacterium sp., Acinetobacter sp., Corynebacterium sp., Thiobacillus ferrooxidans, Thiobacillus intermedia, Thiobacillus Shewanella sp., Micrococcus cinneabareus, Micrococcus sp., Bacillus sulfasportare, bacillus sp., Fungi, White wood rot fungi, Phanerochaete chrysosporium Phanerochaete sordida, Trametes trogii, Tyromyces palustris, white wood rot fungal sp., Streptomyces fradiae, Streptomyces globisporus, Streptomyces sp., Saccharomyces cerrevisiae, Candida sp., Cryptococcus albidus, Algae, sp. of the genus Thiobacillus, such as Thiobacillus denitrificanus, and any combination therein.

30. The method of claim 28, wherein said sulfur consuming bacteria is at least one of non-pathogenic, non-opportunistic, low-virulence factor, and any combination therein.

31. The method of claim 1, further comprising at last one nutrient in said aqueous solution.

32. The method of claim 1, further comprising in said aqueous solution at least one selected from the group consisting of: a phosphate, ammonium hydroxide, sulfur, iron, a carbon compound, and any combination therein.

33. The method of claim 1, wherein the pH in aqueous solution is between 6 and 10.

34. The method of claim 1, wherein the pH in aqueous solution is between 8 and 9.

35. The method of claim 1, wherein said aqueous solution comprises a base or a buffer.

36. The method of claim 1, further comprising in said aqueous solution at least one selected from the group consisting of hydroxide, bi-carbonate, magnesium, and any combination therein.

37. The method of claim 1, wherein the temperature of said aqueous solution is between 17 and 70° C.

38. The method of claim 1, wherein the temperature range of said aqueous solution is 5 to 45° C.

39. The method of claim 1, further comprising at least one of heating and cooling of said aqueous solution.

40. The method of claim 1, wherein said ABR(s) or said ABR Cluster is insulated.

41. The method of claim 1, wherein said aqueous solution comprises an O₂ concentration of 40 percent or less.

42. The method of claim 1, further comprising
gas/liquid separation means, wherein
the effluent aqueous solution from said ABR(s) is at least partially separated into a gas and a liquid.

43. The method of claim 42, wherein said liquid returns to said aqueous solution.

44. The method of claim 42, further comprising a means of bypassing said gas/liquid separation means with said effluent aqueous solution, and wherein
said effluent aqueous solution is returned to said aqueous solution.

45. The method of claim 44, wherein said ABR produces O₂ and the O₂ in said gas is at least partially separated from said gas by gas separation means.

46. The method of claim 45, wherein said gas separation means is at least one of: membrane, vacuum swing adsorption, pressure swing adsorption, and cryogenic distillation.

47. The method of claim 1, wherein the concentration of O₂ is reduced in said aqueous solution and at least one of S and N₂ is reduced enough to facilitate in each ABR or ABR Cluster the production of H₂ instead of O₂.

48. The method of claim 47, further comprising at least one ABR producing O₂.

49. The method of claim 48, wherein at least a portion of said O₂ is used as an oxidant along with the combustion of said H₂ as a fuel to provide power to or heat to said ABR(s).

50. The method of claim 47, wherein at least a portion of said H₂ and at least a portion of said O₂ is used to provide power for at least one of
the separation of O₂ from said ABR(s) vent or said gas,
the separation of H₂ from said ABR(s) vent or said gas, and
the generation of photons for said ABR(s).

51. The method of claim 42, further comprising the treatment of said liquid in an FBR, wherein at least one of:
NO₂ or NO₃ is converted into N₂, and
S_{X} is converted into sulfur within the biomass of sulfur consuming bacteria.

52. The method of claim 51, wherein said FBR comprises denitrifying bacteria.

53. The method of claim 52, wherein said denitrifying bacteria is at least one of non-pathogenic, non-opportunistic, low-virulence factor, and any combination therein.

54. The method of claim 51, wherein said FBR comprises at least one selected from the group consisting of: gram-negative bacteria from the beta or gamma subgroup of Proteobacteria, obligate autotrophs, Thioalkalovibrio, strain AL-2, Thioalkalobacter, alkaliphilic heterotrophic bacteria, Pseudomonas strain ChG 3, Rhodococcus erythropolis, Rhodococcus rhodochrous, Rhodococcus sp., Nocardia erythropolis, Nocardia corrolina, other Nocardia sp., Pseudomonas putida, Pseudomonas oleovorans, Pseudomonas sp., Arthrobacter globiformis, Arthobacter Nocardia paraffinae, Arthrobacter paraffineus, Arthrobacter citreus, Arthrobacter luteus, Arthrobacter sp., Mycobacterium vaccae JOB, Mycobacterium sp., Acinetobacter sp., Corynebacterium sp., Thiobacillus ferrooxidans, Thiobacillus intermedia, Thiobacillus Shewanella sp., Micrococcus cinneabareus, Micrococcus sp., Bacillus sulfasportare, bacillus sp., Fungi, White wood rot fungi, Phanerochaete chrysosporium, Phanerochaete sordida, Trametes trogii, Tyromyces palustris, white wood rot fungal sp., Streptomyces fradiae, Streptomyces globisporus, Streptomyces sp., Saccharomyces cerrevisiae, Candida sp., Cryptococcus albidus, Algae, sp. of the genus Thiobacillus, such as Thiobacillus denitrificanus, and any combination therein.

55. The method of claim 51, wherein said sulfur consuming bacteria is at least one of: non-pathogenic, non-opportunistic, low-virulence factor, and any combination therein.

56. The method of claim 51, further comprising separation of sulfur from said sulfur consuming bacteria.

57. The method of claim 42, further comprising a means of liquid/solids separation, wherein
said liquid is mostly separated into an aqueous portion and a solids portion, and wherein
the solids portion comprises algae.

58. The method of claim 57, wherein at least a portion of said liquid is returned to said aqueous solution.

59. The method of claim 57, further comprising a means of liquid/solids separation, wherein the amount of water with said algae is reduced in said solids portion.

60. The method of claim 57 or 59, wherein said liquid solids separation comprises at least one selected from the group consisting of a: cationic coagulant, a quaternized cationic coagulant, cationic polyacrylamide, quaternized polyacrylamide, poly(DADMAC), poly(DADMAC) comprising a molecular weight of at least 1,000,000, poly(epi-DMA), poly(epi-DMA) comprising a molecular weight of at least 500,000, chitosan cationic polymer, quaternized chitosan polymer, starch cationic polymer, quaternized starch polymer, and any combination therein.

61. The method of claim 1, wherein said ABR(s) comprise a media.

62. The method of claim 1, wherein the algae is used as at least one selected from the group consisting of a: protein in food applications, animal feed, hydrocarbon oil(s), combustion, fertilizer, and any combination therein.

63. The method of claim 62, wherein at least a portion of said algae or said hydrocarbon oil is combusted to generate electricity.

64. The method of claim 63, wherein at least a portion of said electricity is used to generate photons and at least a portion of the photons are used in at least one of said ABR(s).

65. The method of claim 1, further comprising gas from the acidification of a metal-CO₃.

66. The method of claim 2, further comprising gas from the acidification of a metal-NO₂ or a metal-NO₃.

67. The method of claim 65 or 66, wherein said acidification comprises sulfuric acid or carbonic acid.

68. The method of claim 65, wherein said metal salt comprises a Group IA or IIA metal.

69. The method of claim 65 or 66, wherein said metal salt comprises at least one selected from the group consisting of: potassium, sodium, magnesium, calcium, and any combination therein.
